(19) United States
(12) Patent Application Publication
(10) Pub. No.: US 2024/0228925 A1
(43) Pub. Date: Jul. 11, 2024

(54) APPARATUS AND METHOD FOR A BIOMIMETIC HUMAN ALVEOLAR LUNG-ON-A-CHIP MODEL

(71) Applicant: The Brigham and Women's Hospital, Inc., Boston, MA (US)

(72) Inventor: Yu Shrike Zhang, Malden, MA (US)

(21) Appl. No.: 18/558,234

(22) PCT Filed: May 2, 2022
(86) PCT No.: PCT/US2022/027234

§ 371 Date: Oct. 31, 2023

Related U.S. Application Data
(60) Provisional application No. 63/183,033, filed on May 2, 2021.

(51) Int. Cl. C12M 3/00; C12M 1/00; C12M 1/42; C12N 9/10

(52) U.S. Cl. CPC C12M 21/08; C12M 23/26; C12M 35/04; C12N 9/1044; C12Y 203/02013

(57) ABSTRACT

An alveolar lung model apparatus, including: an elastic hydrogel disposed within a housing, the hydrogel including a plurality of sacs; and a pressure chamber disposed within the housing and adjacent to the hydrogel, the pressure chamber coupled to the hydrogel such that changes in pressure within the pressure chamber cause deformation of the hydrogel.

### CROSS-REFERENCE TO RELATED APPLICATIONS

The present application is based on and claims priority from U.S. Patent Application Ser. No. 63/183,033, filed on May 2, 2021, the entire disclosure of which is incorporated herein by reference.

### STATEMENT REGARDING FEDERALLY SPONSORED RESEARCH

N/A

### BACKGROUND

The lung, as the major organ of the human respiratory system, is responsible primarily for gas exchanges and thus, directly exposed to the external environment. Lungs are affected by a plethora of pathologies such as asthma, chronic obstructive pulmonary disease (COPD), and infections like influenza, pneumonia, and tuberculosis as well as lung cancer. These pathological manifestations make lung failure one of the leading causes of death globally. More recently, this is further exemplified by the coronavirus disease 2019 (COVID-19) pandemic, which has so far killed more than 6 million people worldwide with total confirmed cases of >500 million.

The lack of reliable and physiologically relevant animal models for human respiratory diseases has led to a critical issue for new drug development as more than 90% of the preclinical studies performed in animals do not predict the outcome of human clinical trials. This has resulted in a lack of progress in drug development in respiratory medicine, with only a handful of new drugs entering clinical use in the last 50 years. In addition, many of the existing cell culture-based models do not replicate the key biological aspects of the human lung and do not adequately reflect the host responses. These models range from simple two-dimensional (2D) cultures of lung cells on polymeric or elastomeric membrane systems to the complex biomimetic lung-on-a-chip microdevices. While each of these more complex models may have advantages over 2D single-cell cultures, collectively they suffer from important limitations such as use of synthetic polymer membranes with nonphysiological stiffness to culture cells and/or lack of mechanical stimulation (inhalation/exhalation process).

### SUMMARY OF THE INVENTION

Accordingly, new systems, methods, and apparatus for providing a human alveolar lung model are desirable.

There is increasing acceptance that the composition and topography of the extracellular matrix (ECM) have major influences on cell functions and regulate cellular responses to various stimuli. As such, ECM features should not be ignored in the design and fabrication of any biologically relevant tissue and disease models. Therefore, there is a clear need for an advanced model system that not only mimics the human lung tissue structurally but also captures its ECM physiology that is important for the cells' and tissues' functional reproduction in vitro. More importantly, there are no satisfactory models of the distal lung (i.e., the alveolar space) that truly reflect the sac shape anatomy to study physiology and pathophysiology.

Thus, disclosed herein are embodiments of a physiological human-based model that successfully reconstructs the microstructure, ECM properties, air-cell interface, and breathing events of the pulmonary alveoli, which are the basic units enabling gas exchanges in the human lung. Embodiments of this model, termed as the “alveolar lung-on-a-chip,” include a three-dimensional (3D) porous hydrogel made of low-stiffness gelatin methacryloyl (GelMA) featuring an inverse opal structure bonded to a compartmentalized polydimethylsiloxane (PDMS) chip device that provides the medium supply, the air-liquid interface (ALI), and the cyclic mechanical movements (FIG. 1). The 3D hydrogel may be based on an inverse opal structure and may have a stiffness similar to the native human distal lung. We showed through multiscale analyses that our 3D GelMA inverse opal structure was better able to maintain the functions of primary human alveolar epithelial cells in a more in vivo-like manner compared with planar models.

One embodiment provides a lung model apparatus, including: an elastic hydrogel disposed within a housing, the hydrogel including a plurality of sacs; and a pressure chamber disposed within the housing and adjacent to the hydrogel, the pressure chamber coupled to the hydrogel such that changes in pressure within the pressure chamber cause deformation of the hydrogel.

Another embodiment provides a method of making a lung model, including: disposing a plurality of beads into an opening in a housing; pouring a hydrogel-precursor into the opening of the housing in an interstitial space of the plurality of beads; crosslinking the hydrogel-precursor to produce a hydrogel; and dissolving the beads to produce an elastic hydrogel structure including a plurality of sacs.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows the mechanics of a breathing human (left) compared to the disclosed alveolar lung-on-a-chip (right), including schematic diagrams showing the distal lung, the breathing cycles, and the in vitro on-chip model of the breathing alveolar lung.

FIG. 2 shows a cross-section of a lung model apparatus through the line A-A′ in FIG. 27, subpanel viii, according to certain constructions of the disclosure.

FIG. 3 shows procedures for fabrication of the alveoli-like 3D gelatin methacryloyl (GelMA) inverse opal structure and formation of the alveolar lung model. (Panel A, subpanels i and ii) Schematics and bright-field optical images showing the fabrication process of the alveoli-like 3D GelMA inverse opal structure: alginate microbeads with uniform sizes (201±12 μm) were first assembled into a cubic close-packed lattice (Left); a 7% (wt/wt) GelMA solution was then infiltrated into the void spaces of the lattice and cross-linked (Center); finally, the alginate microbeads were selectively removed using ethylene diamine tetraacetic acid disodium (EDTA), leaving behind an alveoli-like hydrogel structure (Right). (Scale bar: 100 μm.) Panel A, subpanel ii, Right, Inset shows a scanning electron microscopy image of the interconnecting window between two adjacent sacs. (Scale bar: 20 μm; note the shrinkage of the sac size comparing to optical images was caused by the sample-drying process for scanning electron microscopy imaging.) (Panel A, subpanel iii) Fluorescence confocal images illustrating the GelMA inverse opal hydrogel structure, where GelMA was chemically labeled with FITC: (Left) after infiltration of the GelMA solution into the void spaces of the alginate microbead lattice and cross-linking, (Center) after removal of the alginate microbeads, and (Right) showing a 3D reconstruction of the GelMA hydrogel structure. (Scale bars: 100 μm.) (Panel B, subpanel i) Fluorescence confocal images showing primary human alveolar epithelial cells (hAECs) cultured in the GelMA inverse opal hydrogel structures at 1, 7, and 14 d. Green (left col.), live cells; red (right col.), dead cells. (Scale bar: 100 μm.) (Panel B, subpanel ii) Quantification of viability (Left) and proliferation (Right) of the human alveolar epithelial cells (hAECs) in the GelMA inverse opal hydrogel structures. * P<0.05. (Panel C) Confocal reconstruction view (Left) and sectional view (Right) of the hAECs after culturing for 14 d in the GelMA inverse opal hydrogel structure, in which the fully confluent alveolar epithelium was formed. Green, F-actin; blue, nuclei. (Scale bars: 100 μm.) (Panel D) Confocal micrographs showing the presence of continuous tight junctions of the rim (Left) and bottom (Right) of a sac after culturing for 14 d in the GelMA inverse opal hydrogel structure. White, zonula occludens (ZO-1); blue, nuclei. (Scale bars: 50 μm.) (Panel E) Representative image of an H&E-stained section of the alveolar lung model after 14 d of culture. (Scale bar: 50 μm).

FIG. 4 shows comparisons of the alveolar epithelium formation for hAECs cultured on 3D and 2D substrates of GelMA and PDMS. (Panel A) Scanning electron microscopy images of the hAECs cultured on/in the 3D GelMA inverse opal structure (Upper) and the 2D GelMA hydrogel (Lower) at days 3, 7, and 14. (Scale bars: 25 μm.) (Panel B) Normalized proliferation of hAECs on the two GelMA surfaces. *** P<0.001. (Panel C) Caspase-3/7 fluorescence signal analysis and annexin V-FITC/PI flow cytometry plots of hAECs on the two GelMA surfaces. ** P<0.01. (Panel D) Flow cytometry analyses of annexin V-FITC- and PI-stained hAECs cells at day 14. (Panel E) K-means clusters of the genes for (subpanel i) GO enrichment and (subpanel ii) Kyoto Encyclopedia of Genes and Genomes (KEGG) enrichment analyses of hAECs cultured on 2D PDMS, on 2D GelMA, and in 3D GelMA for 14 d. (Panel F) Heat maps showing differential gene expression relating to (subpanel i) ECM-receptor interactions and (subpanel ii) TNF signaling pathway of hAECs cultured on 2D PDMS, on 2D GelMA, and in 3D GelMA for 14 d. Abbreviations: a.u.: any unit; PI3K-Akt: phosphatidylinositol 3-kinase-protein kinase B; HIF-1α: hypoxia-inducible factor-α.

FIG. 5 shows construction of the alveolar lung-on-a-chip model. (Panel A) Schematic representing the alveolar lung-on-a-chip (Upper) and photograph of a device without the GelMA inverse opal structure to show the underlying fluidic channels (Lower). (Scale bar: 5 mm.) (Panel B) Fluorescence images showing the viability of the hAECs cultured in the chips without and with the breathing events. Green (left), live cells; red (center), dead cells; (right) brightfield. (Scale bar: 100 μm.) (Panel C) Micrographs showing the expansion of the sacs under a strain of 8%. (Scale bar: 100 μm.) (Panel D) Images of H&E-stained sections showing the epithelium formation in the chips without (control, left) and with (right) the breathing events. (Scale bar: 50 μm.) (Panel E) ZO-1 staining showing the tight junction formation of the epithelium in the chips without and with the breathing events. White, ZO-1; blue, nuclei. (Scale bar: 50 μm.) (Panel F) The quantified levels of secreted cytokines IL-8, IL-6, IL-1B, MCP-1, and GM-CSF by hAECs cultured in the chips without and with the breathing events. All analyses were performed at 14 d of culture.

FIG. 6 shows the effects of smoking and SARS-Cov-2 pseudoviral infection on the alveolar lung-on-chip model. (Panel A) Schematic diagrams showing the smoking alveolar lung-on-a-chip. (Panel B) Live/dead staining of the hAECs in the alveolar lung-on-a-chip devices before and after smoking. Green, live cells; red, dead cells. (Scale bar: 100 μm.) (Panel C) Quantification of cell viability for hAECs. Control: without breathing and smoking; breathing: with breathing but without smoking; and breathing+smoking: with breathing and smoking. (Panel D) Confocal fluorescence images showing the effects of smoking on tight junctions of the alveolar epithelium, revealed by ZO-1 staining. White, ZO-1; blue, nuclei. (Scale bar: 100 μm.) (Panel E) Confocal fluorescence images showing induction of oxidative stress with smoking. Green: 4-HNE; blue, nuclei. (Scale bar: 100 μm.) (Panel F) The quantified levels of secreted cytokines IL-8, IL-6, IL-1β, MCP-1, and GM-CSF by hAECs cultured in the chips. All analyses were performed at 14 d of culture, and smoking was conducted for 75 min. (Panel G) Fluorescence confocal images showing the expression of ACE2 receptors by hAECs in the GelMA inverse opal structures at day 14. Red, ACE2 receptors; blue, nuclei. (Scale bar: 50 μm.) (Panel H) Fluorescence microscopic images showing the live/dead staining for hAECs in the GelMA inverse opal structures after (subpanel i) SARS-COV-2 pseudoviral particle (pCoV-VP) infection without antiviral drugs, (subpanel ii) pCoV-VP infection in the presence of amodiaquine (5 μM), (subpanel iii) pCoV-VP infection in the presence of remdesivir (10 μM), and (subpanel iv) pCoV-VP infection in the presence of hydroxychloroquine (40 μM). Green, live cells; red, dead cells. (Scale bar: 100 μm.) (Panel I) MTS assay showing metabolic activities of hAECs in the GelMA inverse opal structures after pCoV-VP infection in the absence and presence of antiviral drugs. *P<0.05; * P<0.01; * P<0.001.

FIG. 7 shows compressive modulus values of GelMA hydrogel crosslinked with only Irgacure 2959 (single-crosslinking) and with both Irgacure 2959 and microbial transglutaminase (mTG) (double-crosslinking).

FIG. 8 shows swelling behaviors and degradation profiles of 2D and 3D GelMA hydrogels. (Panel A) Swelling ratios (in PBS for 24 h) showing of 2D GelMA and 3D GelMA hydrogels fabricated with different crosslinking methods. * p<0.05. (Panel B) Degradation profiles (in PBS expedited by the presence of 2 U mL⁻¹ of collagenase I) of 3D GelMA hydrogels with different crosslinking methods.

FIG. 9 shows 3D ColMA inverse opal structure (Panel A) before and (Panel B) after the removal of alginate microbeads. Scale bar, 100 μm.

FIG. 10 presents scanning electron microscopy images showing the cross-section of the 3D GelMA scaffold. (Left) Scanning electron microscopy image of the cross-section of the 3D GelMA structure and (right) the enlarged view of the red dotted frame area on the left. Scale bar, (left) 100 μm and (right) 20 μm. Note the shrinkage of the sac size comparing to optical images was caused by the sample-drying process for scanning electron microscopy imaging.

FIG. 11 shows (Panel A) The electrospraying device for the fabrication of alginate microbeads. (Panel B) (subpanel i) The formation of the alginate microbeads. (subpanel ii) SEM image of an alginate microbead. (subpanel iii) X-ray energy dispersive spectroscopy (EDS) mapping of a Ca²⁺-crosslinked alginate microbead. Scale bars: 25 μm.

FIG. 12 shows the effect of alginate concentration on the resulting microbeads. Optical micrographs showing the obtained alginate microbeads when the alginate concentrations were (Panel A) 0.5% (w/w), (Panel B) 1.5% (w/w), (Panel C) 2.5% (w/w), and (Panel D) 3.5% (w/w). Scale bar, 100 μm. (Panel E) Plot showing the quantified diameters as a function of alginate concentration.

FIG. 13 shows the effect of voltage on the resulting microbeads. Optical micrographs showing the obtained alginate microbeads when the voltages were (Panel A) 9 kV, (Panel B) 11 kV, (Panel C) 13 kV, (Panel D) 15 kV, and (Panel E) 17 kV. Scale bar, 100 μm. (Panel F) Plot showing the quantified diameters as a function of voltage.

FIG. 14 shows the effect of nozzle diameter on the resulting microbeads. Optical micrographs showing the obtained alginate microbeads when the nozzle diameters were (Panel A) 140 μm, (Panel B) 210 μm, and (Panel C) 330 μm. Scale bar, 100 μm. (Panel D) Plot showing the quantified diameters as a function of nozzle diameter.

FIG. 15 shows the effect of flow rate on the resulting microbeads. Optical micrographs showing the obtained alginate microbeads when the flow rates were (Panel A) 5 mm h⁻¹, (Panel B) 10 mm h⁻¹, (Panel C) 15 mm h⁻¹, and (Panel D) 20 mm h⁻¹. Scale bar, 100 μm. (Panel E) Plot showing the quantified diameters as a function of flow rate.

FIG. 16 shows the effect of ring diameter on the resulting microbeads. Optical micrographs showing the obtained alginate microbeads when the flow rates were (Panel A) 7 cm, (Panel B) 8 cm, (Panel C) 9 cm, and (Panel D) 10 cm. Scale bar, 100 μm. (Panel E) Plot showing the quantified diameters as a function of ring diameter.

FIG. 17 shows GelMA inverse opal structures fabricated by templating alginate microbeads with different yet uniform diameters. (Panels A, B, and C) Brightfield images showing (subpanels i) the close-packed microbeads of 325.9±10.5, 199.6±1.1, and 156.3±2.7 μm in diameters, respectively, and (subpanels ii) the corresponding GelMA inverse opal structures after alginate microbead removal. Scale bar, 100 μm.

FIG. 18 shows GelMA scaffold with nonuniform pore sizes. (Panel A) Bright-field images showing the template made of randomly mixed alginate microbeads containing three different sizes (˜200, ˜250, and ˜300 μm) and (Panel B) the corresponding GelMA structure after microbead removal. Scale bar, 100 μm.

FIG. 19 shows characterizations of the microbead removal. SEM images and corresponding EDS mapping of GelMA inverse opal hydrogel structures (Panel A) before and (Panel B) after the microbead removal. Scale bar, 50 μm. The inset in (Panel A, subpanel i) shows the overlaid distribution of the Ca²⁺ in the scaffold containing the alginate microbeads. Scale bar, 50 μm. Note the shrinkage of the sizes comparing to optical images was caused by the sample-drying process for scanning electron microscopy imaging.

FIG. 20 shows hAECs at different depths in a single layer of the sac within a 3D GelMA inverse opal structure for 14 days. (Panel A) Fluorescence confocal images showing the hAECs cultured in a GelMA inverse opal hydrogel structure with (subpanel i) projection view, as well as (subpanels ii-v) views of the different depths (from middle and bottom). (Panel B) Morphologies of the hAECs after culturing in a GelMA inverse opal structure with (subpanel i) projection view, as well as (subpanels ii-v) views of the different depths (from middle and bottom). Note that in (subpanels i, iv, and v) the ventilation windows connecting the adjacent sacs are clearly visible. Scale bar, 100 μm.

FIG. 21 shows F-actin/nuclei staining of the hAECs in the 3D GelMA structures at day 21. Scale bar, 50 μm.

FIG. 22 shows morphologies of hAECs on the surfaces of 2D GelMA structure. (Panels A, B) Confocal projection and reconstruction views for cells cultured for 7 days and 14 days, respectively. Green, F-actin; blue, nuclei. (Panel C) Bright-field image showing the oftentimes incomplete surface coverage (marked with stars, ★) and local aggregations (arrows) even after 14 days of culture for hAECs on the flat GelMA surface. Scale bar, 100 μm.

FIG. 23 shows quantification of proliferation of hAECs on the 2D GelMA surfaces and in 3D GelMA structures over the culture period of 14 days. * p<0.05; ** p<0.01; *** p<0.001.

FIG. 24 shows apoptosis of hAECs. Fluorescence images showing caspase-3/7 staining for hAECs cultured on the surface of 2D GelMA hydrogel block (2D GelMA) and in the alveolar-like GelMA inverse opal structure (3D GelMA). Scale bar, 100 μm.

FIG. 25 shows flow cytometry analyses of annexin V-FITC- and PI-stained hAECs on 2D GelMA surfaces and in 3D GelMA structures at day 22.

FIG. 26 shows the differential gene expressions of hAECs cultured on 2D surfaces of GelMA slabs and PDMS slabs, as well as in the 3D GelMA inverse opal structures. (Panel A) Scatter plots of differentially expressed genes for comparisons of 3D GelMA versus 2D GelMA, 3D GelMA versus 2D PDMS, and 2D GelMA versus 2D PDMS. The plots are color-coded, where red stands for up-regulated genes and blue for down-regulated genes. (Panel B) Venn diagram showing the number of genes differentially expressed among the three groups. (Panel C) KEGG pathway analyses showing enriched pathways of differential expressed genes for comparisons of 3D GelMA versus 2D GelMA, 3D GelMA versus 2D PDMS, and 2D GelMA versus 2D PDMS. (Panels D, E) gene ontology (GO) enrichment analyses and KEGG pathway analyses, respectively, of expression trends of genes identified with K-means clusters for hAECs cultured under the three different conditions.

FIG. 27 shows a schematic representation of the fabrication process of the alveolar lung-on-a-chip.

FIG. 28 shows computational simulation of airflows for 3D and 2D structures during a single breathing cycle. (Panels A, B) 3D distribution of air streamlines during the stretching of (Panel A) a 3D inverse opal alveolar structure and (Panel B) a control planar structure (at the bottom of the cube) at different time points. (Panels C, D) Air flow vectors showing the direction of air flow during the stretching of (Panel C) the 3D inverse opal alveolar structure and (Panel D) the control planar structure (at the bottom of the cube). The red vectors represent the air inflow and blue vectors represents the air outflow. (Panels E, F) Distributions of air velocities during the stretching of (panel E) the 3D inverse opal alveolar structure and (Panel F) the control structure, at different time points.

FIG. 29 shows biomolecule diffusion in the 3D GelMA inverse opal scaffold. (Panels A, B), Time-lapse fluorescence images of diffused (Panel A) FITC from (subpanels i to vi) 15, 30, 45, 60, 75, and 90 min, respectively and (Panel B) FITC-dextran (Mw=10 kDa) from (subpanels i to vi) 30, 60, 90, 120, 150, and 180 min, respectively. The red arrows represent the diffusion direction of the FITC or FITC-dextran, and the red dotted boxes enclose the 3D GelMA inverse opal structures. Scale bar, 1 mm. (Panels C, D) Quantified mean gray value intensities of the representative images at the different time points, for (Panel C) FITC and (Panel D) FITC-dextran.

FIG. 30 shows apoptotic activities of hAECs before and after the application of the cigarette smoke. (Panel A) Fluorescence microscopic images showing caspase-3/7-stained hAECs on 2D GelMA surfaces (upper row) before and (bottom row) at 24 h after the application of smoke. (Panel B) Fluorescence microscopic images showing caspase-3/7-stained hAECs on 3D GelMA inverse opal structures (upper row) before and (bottom row) at 24 h after the application of smoke. Scale bar, 100 μm. (Panel C) Quantification of fluorescence intensity signals of the hAECs stained with caspase-3/7 on 2D GelMA surfaces and in 3D GelMA structures before and at 24 h after the application of smoke. * p<0.05. (Panel D) Quantification of cell metabolic activities of the hAECs on 2D GelMA surfaces and in 3D GelMA structures before and at 24 h after the application of smoke by the MTS assay.

FIG. 31 shows SARS-COV-2 pseudoviral infection and treatment efficacies on the alveolar lung-on-a-chip model. (Panel A) Fluorescence confocal images showing the expression of ACE2 receptors by hAECs in the GelMA inverse opal structure at day 14. Red, ACE2 receptors; blue, nuclei. (Panel B) Fluorescence microscopic images showing mcherry-expressing pCOV-VP-infected hAECs in the GelMA inverse opal structures in the absence of antiviral drug (pCoV-VP) as well as the presence of 5-μM amodiaquine (pCoV-VP+AQD), 10-μM remdesivir (pCoV-VP+RDV), or 40-μM hydroxychloroquine (pCoV-VP+HCQ). (Panel C) Fluorescence microscopic images showing live/dead staining of hAECs in the GelMA inverse opal structures after pCOV-VP infection without antiviral drugs (pCoV-VP) as well as the presence of 10-μM remdesivir (pCoV-VP+RDV) or 40-μM hydroxychloroquine (pCoV-VP+HCQ). Green, live cells; red, dead cells. Scale bars, 50 μm.

FIG. 32 presents images recreating the alveoli-capillary interface in the GelMA inverse opal structure. (Panel A) Confocal reconstruction images showing HUVECs grown in the GelMA framework at day 24. (Panel B) Projection views of confocal images showing the potential formation of the microvascular network by the HUVECs at different locations. Green, F-actin; blue, nuclei. Scale bar, 100 μm.

FIG. 33 shows the non-small cell lung cancer (NSCLC) model in the GelMA inverse opal structures. (Panel A) Fluorescence confocal images showing the A549 cells cultured in the GelMA inverse opal structures at days 1, 7, and 14. Green, live cells; red, dead cells. Scale bar, 100 μm. (Panel B) Proliferation of the A549 cells in the GelMA inverse opal structures. * p<0.05; ** p<0.01. (Panel C) (left) Confocal sectional view and (right) reconstruction view of A549 cells after culturing for 14 days in the GelMA inverse opal structure, in which fully confluent cell layers were formed. Green, F-actin; blue, nuclei. Scale bar, 100 μm. (Panel D) Representative image of a H&E-stained section of the NSCLC model after 14 days of culture. Scale bar, 50 μm.

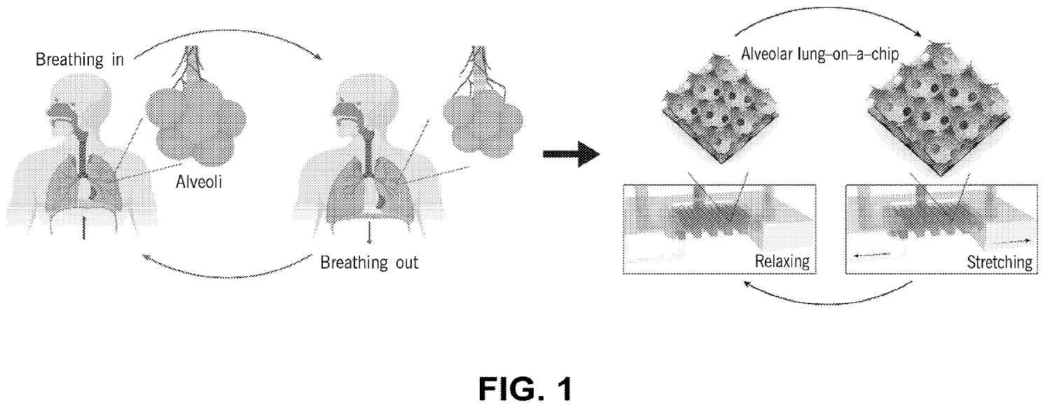

### DETAILED DESCRIPTION

In accordance with some embodiments of the disclosed subject matter, mechanisms (which can include systems, methods, and apparatus) for providing a human alveolar lung model are provided.

Disclosed herein are embodiments of a physiologically relevant model of the human pulmonary alveoli. Embodiments of the disclosed alveolar lung-on-a-chip platform include a three-dimensional porous hydrogel made of GelMA with an inverse opal structure, bonded to a compartmentalized polydimethylsiloxane (PDMS) chip. The inverse opal hydrogel structure features well-defined, interconnected pores with high similarity to human alveolar sacs. By populating the sacs with primary hAECs, functional epithelial monolayers are readily formed. Cyclic strain is integrated into the device to allow biomimetic breathing events of the alveolar lung, which, in addition, makes it possible to investigate pathological effects such as those incurred by cigarette smoking and severe acute respiratory syndrome coronavirus 2 (SARS-CoV-2) pseudoviral infection. Furthermore, when the hydrogel is seeded with suitable cell types the model can be used to study conditions such as lung cancer. Our study demonstrates a unique method for reconstitution of the functional human pulmonary alveoli in vitro, which is expected to pave the way for investigating relevant physiological and pathological events in the human distal lung.

Importantly, the GelMA inverse opal structure has a high similarity to the native human alveolar sacs in that they possess both the sac-like pores and the interconnecting windows between the sacs, in addition to a stiffness close to the native human lung. Due to its 3D nature, it is possible to construct ˜7,050 alveoli within an 8×10×3-mm³ model to allow faithful physiological emulation. Primary hAECs were populated on the surfaces of the sacs to form the monolayer epithelium. The alveolar lung model was further subjected to cyclic inhalation/exhalation movements, as well as used to investigate the effects of cigarette smoke and SARS-COV-2 pseudoviral infection. Our study provides a unique method for reconstitution of functional alveolar lung-on-a-chip in vitro, potentially paving the way for investigating a range of pathophysiology of the human distal lung, such as infectious diseases affecting the alveolar space including COVID-19.

The present disclosure provides various embodiments of a lung model apparatus as well as methods of making and using such a model. The following provides a summary of embodiments of the apparatus and how it may be formed, whereas additional variations and details may be found throughout the remainder of the disclosure. The lung model may include an elastic hydrogel disposed within a housing, where the hydrogel may include a plurality of sacs lined with cells which mimic the alveoli of the lung. As disclosed herein, the sacs are formed in the hydrogel by casting the hydrogel-precursor within an interstitial space of an array of particles (e.g. alginate beads), crosslinking the hydrogel-precursor into the hydrogel, and subsequently removing the beads (e.g. by dissolving the alginate by introducing chelator) to leave the alveolar sac-like structures in the hydrogel. In embodiments such as those disclosed herein in which the particles are spherical beads, the interstitial space includes the empty spaces between adjacent beads.

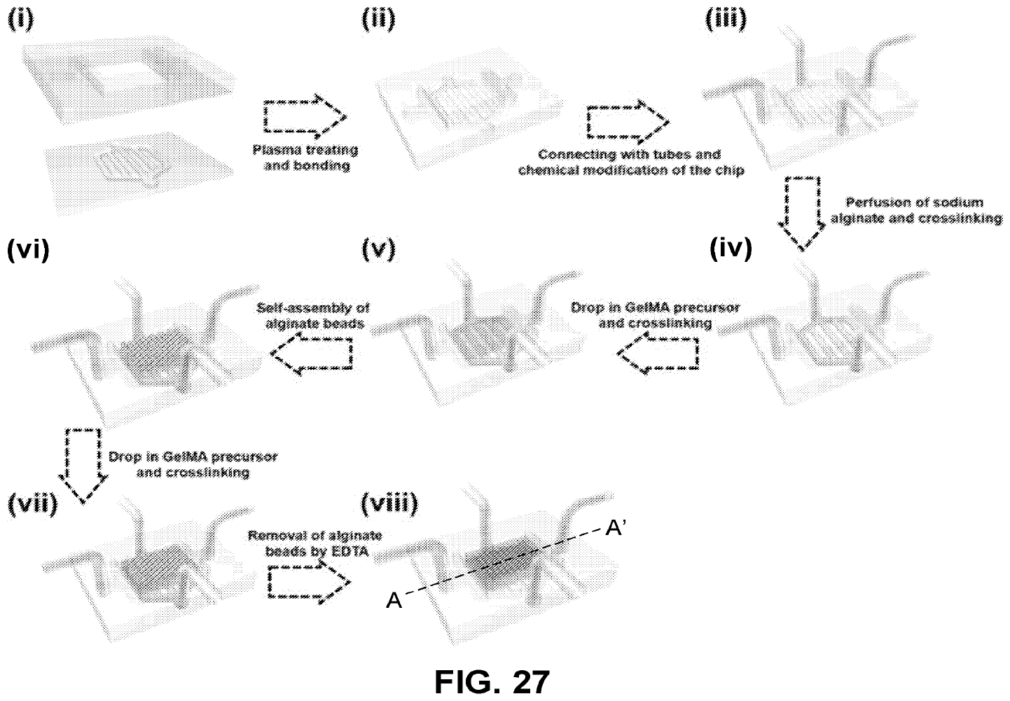

As shown in FIG. 27, subpanel i, the approximately planar housing can be formed by combining a base with an upper portion, one or both of which may be cast from a material such as PDMS in molds made from a material such as laser-cut poly(methyl methacrylate) (PMMA), SU-8, or other material that is suitable for molding. In addition to PDMS, the housing, or chip, may also be made of PMMA or other suitable thermoplastic material. The base may include one or more fluidic channels for carrying cell culture fluid adjacent to the finished hydrogel; although the examples herein depict five fluidic channels, in various embodiments the base may include any suitable number of such channels (e.g. between 1-100) of any suitable dimension for providing sufficient fluid to the hydrogel. After each piece is separately formed, the base and upper portion may be joined to one another using plasma treatment, as described herein. Alternatively, other mechanisms for joining the base and upper portion may be used such as physically clamping the components together.

The upper portion of the base may include an opening (e.g. a square or rectangular opening) that is aligned with the fluidic channels in the final assembly (FIG. 27, subpanels i, ii). The upper portion may also include at least one pressure chamber adjacent to the opening, where the pressure chamber may extend adjacent to at least one side of the central opening of the housing, separated from the opening by a wall portion that is sufficiently flexible so as to resiliently move as a result of changes of pressure within the pressure chamber. The sides of the opening adjacent to the pressure chamber are adhered to the hydrogel (e.g. using plasma treatment) so that pressure decreases within the pressure chamber, which cause the wall to retract away from the opening, stretch the hydrogel toward the side of the opening.

In some embodiments the opposite side of the hydrogel may be adhered to the upper portion of the housing on the opposite side of the opening such that the pulling force from the contracting pressure chamber stretches the hydrogel in the plane of the housing, mimicking the expansion of the chest cavity which expands the alveoli and draws air into the lungs. In particular embodiments the hydrogel may have a variable stiffness, which may depend on the type of lung disease or condition being emulated, and may have a modulus ranging from between about 1 kPa to about 30 kPa and, in particular, for emulating normal lung tissue may have a Young's modulus of about 6 kPa. In various embodiments, the stiffness of the final hydrogel structure may be set through the adjustment of one or more parameters, including by varying the percentage of hydrogel protein (e.g. GelMA) and/or by varying the crosslinking density such as by changing either the crosslinker concentration (which in the case of photo-crosslinking it would be photoinitiator concentration or/and light intensity) or crosslinking time, or both.

In certain embodiments, the upper portion may include two pressure chambers, on opposite sides of the opening, which are attached to the hydrogel such that decreases in pressure in the pressure chambers increases the expansive force by pulling on both sides of the hydrogel. In still other embodiments, pressure chambers may be placed on 3, 4, or other number of sides of the opening (e.g. depending on the shape of the opening) to increase the amount and the direction of application of the force applied to the hydrogel. In various embodiments, the one or more pressure chambers may be coupled to pressure sources which can supply either or both of positive or negative pressure. In certain embodiments, the changes in pressure may be applied cyclically, for example in a range of 0.01 Hz to 10 Hz, or in particular at a rate of 0.2 Hz to mimic typical rates of breathing in humans. In some embodiments the one or more fluidic channels may be coupled to a source of fluid such as cell culture media or blood which is circulated through the fluidic channels adjacent to the hydrogel. The fluid then permeates the hydrogel and provides nutrients to the cells adhered to the hydrogel.

After the base and upper portion of the housing a bonded together, one or more connecting tubes may be coupled to the pressure chamber(s) and fluidic channels (FIG. 27, subpanel iii). In addition, the PDMS material making up the housing may be modified for bonding with the hydrogel material, as described herein. To prepare the housing to receive the hydrogel, the fluidic channels may be filled with a material such as alginate which may be crosslinked by the addition of calcium (see FIG. 27, subpanel iv). A base layer of hydrogel (e.g. a gelatin-based material such as GelMA) may be deposited over the alginate-filled fluidic channels and crosslinked using procedures such as those disclosed herein (see FIG. 27, subpanel v). Next, an array of dissolvable beads (e.g. alginate beads) may be formed on the base layer of hydrogel within the opening in the housing (see FIG. 27, subpanel vi), where the array may be multiple layers of beads deep; in some embodiments the beads may be 3 layers deep although there may be any number of layers and the total number of layers may range from 1-10 or higher. As disclosed herein, the beads may range in size from about 150 μm to about 325 μm and may be all approximately the same size or may be a combination of two or more sizes. Nevertheless, in various embodiments the beads may effectively be any size and may be as small as 10 μm (or smaller) or as large as 500 μm-1000 μm (or larger). In some embodiments the beads self-assemble into an array that resembles the microscopic structure of an opal, with the result that the hydrogel that is formed around the beads takes on what is referred to herein as an “inverse opal” structure.

The array of dissolvable beads is then immersed in hydrogel-precursor which is subsequently crosslinked (see FIG. 27, subpanel vii), e.g. using UV or visible light (e.g. when a photoinitiator is present in the photoactive hydrogel-precursor) and/or other chemistry (e.g. when the hydrogel-precursor includes a protein biomaterial such as gelatin and an enzyme such as mTG), as disclosed herein. In general, the chemical crosslinking agents can be any type that allows crosslinking of the particular hydrogel material that is used and any general chemistry would work such as crosslinking by glutaraldehyde or genipin. Certain crosslinking agents such as glutaraldehyde are not biocompatible and are therefore less desirable for certain uses, while other crosslinking agents such as mTG and genipin are biocompatible and therefore can be applied and/or activated in the hydrogel even in the presence of live cells (e.g. vascular cells) without harming the cells. Finally, the dissolvable material that makes up the beads and the filler in the fluidic channels is dissolved; for alginate, a chelator such as EDTA may be added to cause the material to dissolve (see FIG. 27, subpanel viii), as disclosed herein. The structure that remains is a hydrogel that is adjacent to the fluidic channels and includes a number of cavities, or sacs, that represent open spaces within the hydrogel matrix. Many or most of the sacs are interconnected with adjacent sacs by openings which permit air and liquid to move between the sacs, similar to lung alveoli.

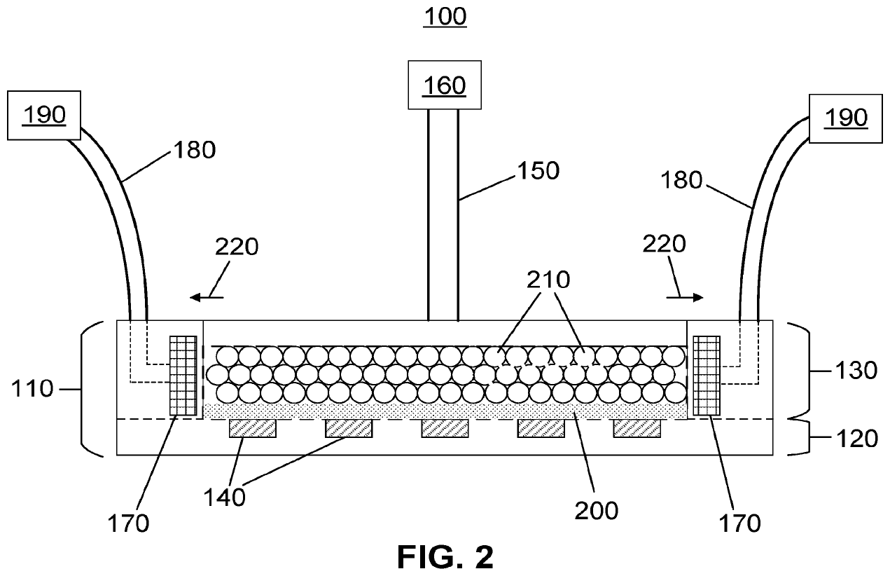

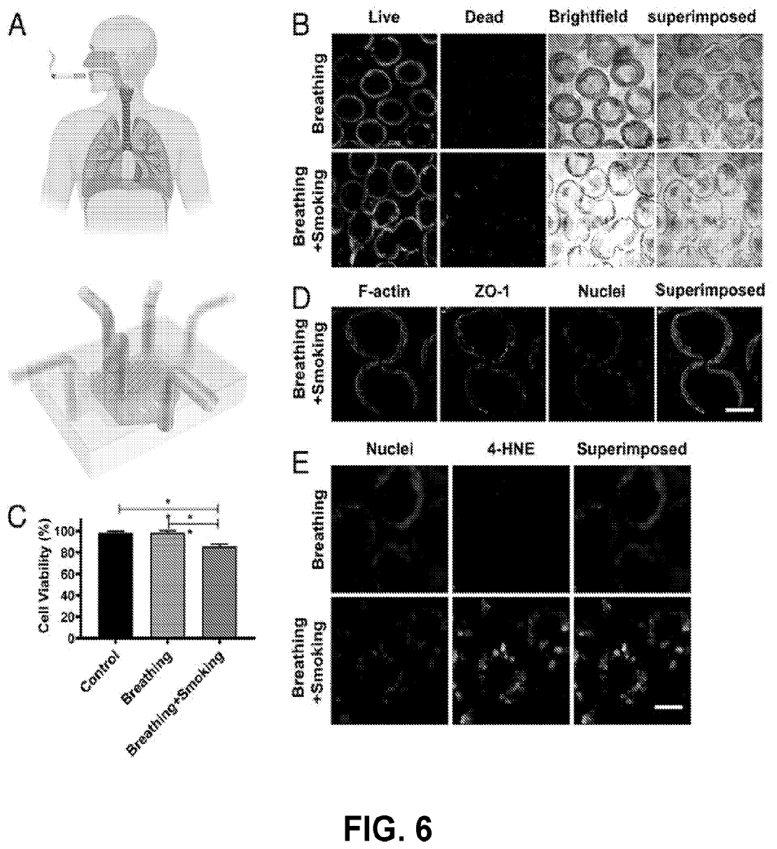

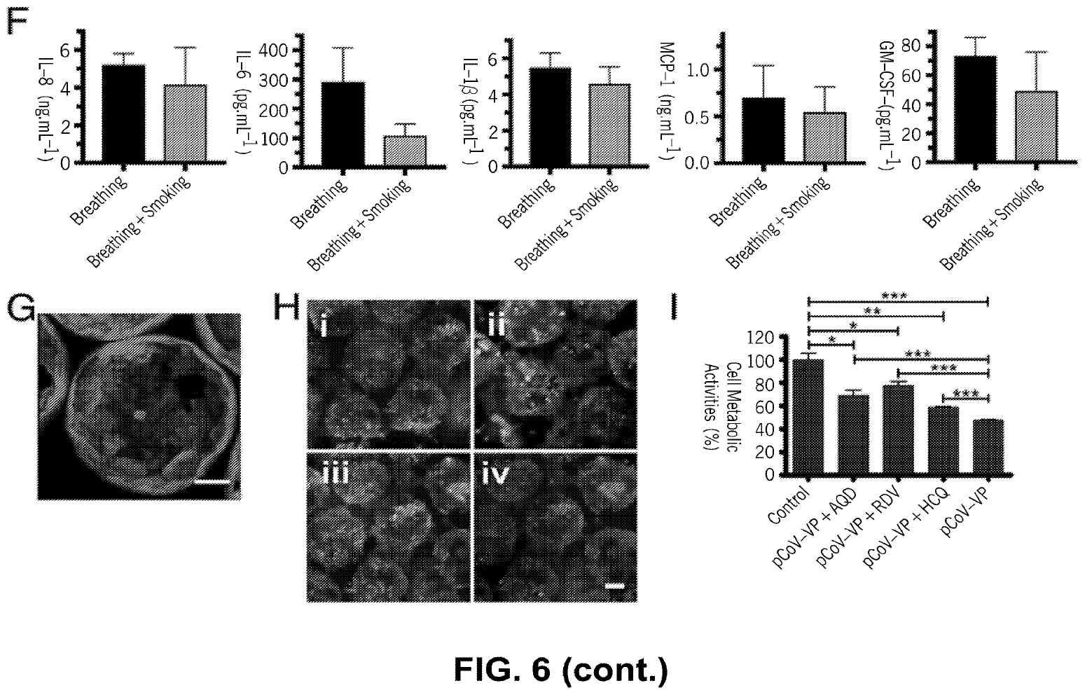

FIG. 2 shows a cross-sectional view of a lung model apparatus 100 according to certain embodiments, as would be viewed for example through the line A-A′ in FIG. 27, subpanel viii. The housing 110 includes the base 120 and the upper portion 130, with multiple fluidic channels 140 formed in the base 120. The fluidic channels 140 are shown connected via fluid tubing 150 to a fluid source 160. Also shown are a pair of pressure chambers 170 coupled via pressure tubing 180 to respective pressure sources 190. In some embodiments in which an airborne material such as cigarette smoke is applied to the hydrogel sacs, additional tubing and/or a cover may be placed over the hydrogel to deliver the airborne material as shown in FIG. 6A.

The hydrogel includes the base layer 200 along with the numerous sacs 210 formed therein, where the sacs 210 may be interconnected to one another by openings at the points of contact between adjacent sacs 210. Decreases in pressure in the pressure chambers 170 cause the walls of the upper portion 130 between the pressure chambers 170 and the hydrogel to pull to the sides (as indicated by arrows 220), which stretches the hydrogel in the plane of the housing and causes the sacs to change their size and/or shape, similar to alveoli of the lung. In various embodiments the sacs may increase from about 1% to about 50% in size as a result of the pressure-based stretching of the hydrogel and in particular embodiments the sacs may increase in size by about 8%; the degree of stretching of the hydrogel can be controlled by a number of factors such as the size and number of pressure chambers, the level of pressure applied to the chambers, and/or the frequency of pressure changes, among other factors. While the examples presented herein show the hydrogel being pulled in a single dimension or direction (e.g. along one axis parallel to the planar housing), in some embodiments the hydrogel may be pulled in two or more directions depending on the number and layout of pressure chambers in the housing.

After the hydrogel has been formed, cells may be added to the hydrogel, where the cells adhere to the surface of the hydrogel and in particular to the sacs, as disclosed herein. In some embodiments the cells may be primary hAECs, although other cell types may be used as well. For example, in some embodiments other cells that can be added to the hydrogel include various structural and functional cells of the distal lung such as endothelial cells (e.g. from umbilical vein), fibroblasts, and immune cells. The timing of when the particular cells are associated with the hydrogel can be varied as well: in various embodiments the hAECs may be added after the hydrogel has been formed, as disclosed herein, and adhere primarily to the surface of the hydrogel including to the surfaces of the sacs; in other embodiments structural and functional cells of the distal lung such as endothelial cells, fibroblasts, and immune cells may be combined with the hydrogel backbone material, or precursor, before it is cast and crosslinked (in which case a biocompatible crosslinking agent is preferred, see above) and the hydrogel may be crosslinked with the cells embedded in the matrix. In various embodiments the hydrogel may be made of a low-stiffness gelatin-based biomaterial such as GelMA, as disclosed herein, although other materials such as collagen methacryloyl may be used instead of, or in addition to, GelMA. In other embodiments, the hydrogel may include laminin, fibrin, hyaluronic acid, or any type of extracellular matrix (ECM) material, including those materials typically found in the distal lung. In various embodiments, the lung model apparatus may be used to study the biology of lung cells under normal conditions and when the cells are subject to various stresses such as exposure to airborne materials that may be brought into the alveoli such as cigarette smoke or SARS-COV-2 virus, as disclosed herein. In some embodiments, the lung model apparatus may be used to reconstitute certain pathologies in vitro such as a model of non-small cell lung cancer (NSCLC).

### EXAMPLES

The following are non-limiting descriptions of exemplary procedures for making and using the lung model system according to embodiments of the disclosure:

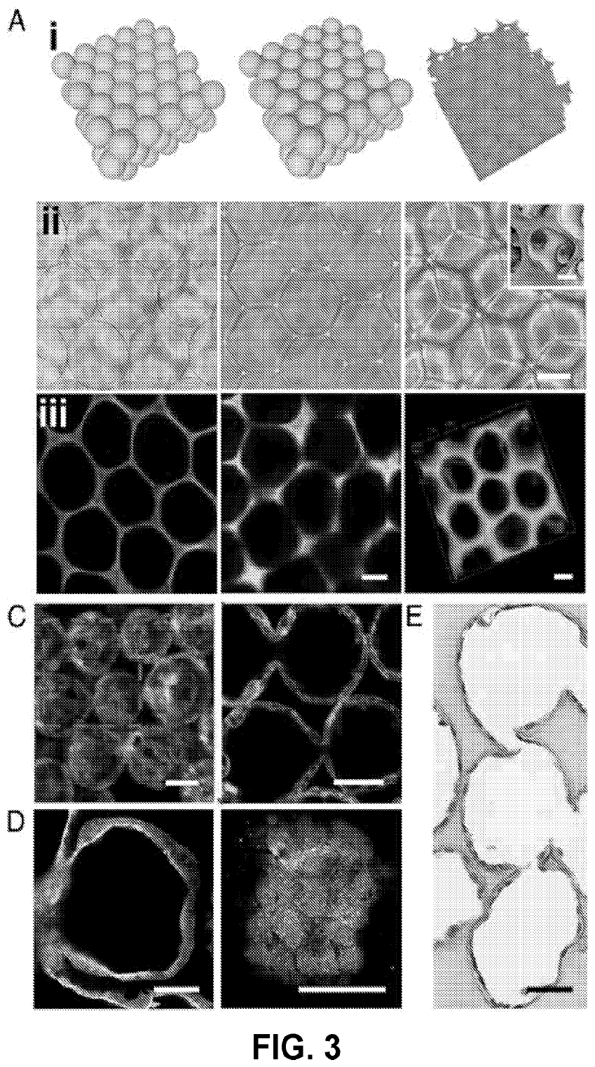

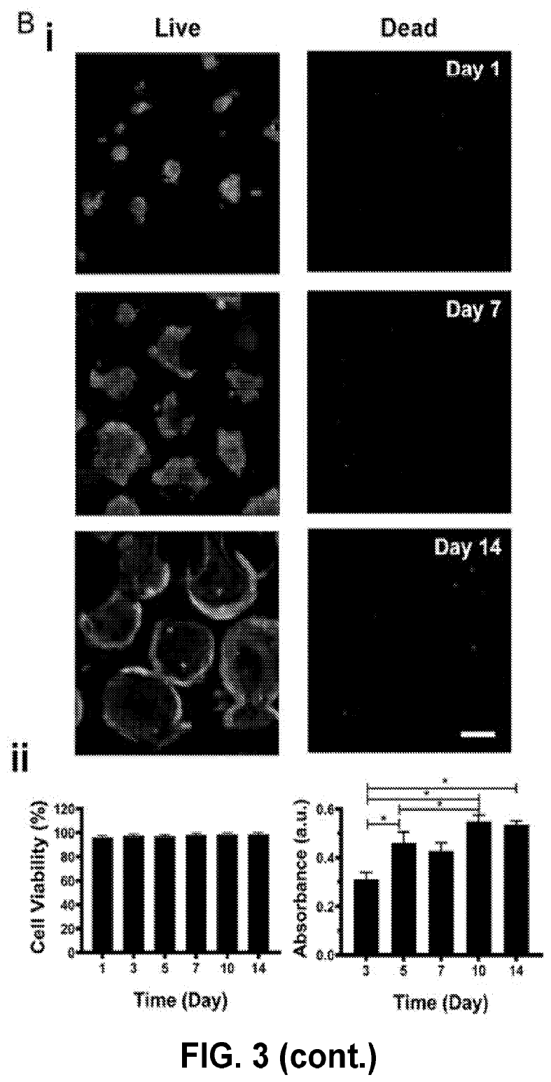

Fabrication of the alveoli-like 3D porous structure and formation of the alveolar epithelium The typical process for the fabrication of the alveoli-like 3D GelMA inverse opal structure is schematically presented in FIG. 3A, subpanel i, where the corresponding optical micrographs are shown in FIG. 3A, subpanel ii. This process largely follows those previously published, with certain modifications. First, alginate microbeads with a uniform size (201±12 μm) were assembled into a cubic close-packed lattice. Then, 7% GelMA solution was infiltrated into the void spaces of the lattice and cross-linked. Finally, after the removal of the alginate microbeads by the 0.01 M ethylene diamine tetraacetic acid disodium (EDTA) solution, an alveoli-like 3D hydrogel with uniform pores and connecting windows was formed. A very limited amount of GelMA solution was able to infiltrate the contact areas between the microbeads prior to their removal, also allowing formation of interconnecting windows between most, if not all, of the adjacent pores to enable effective ventilation.

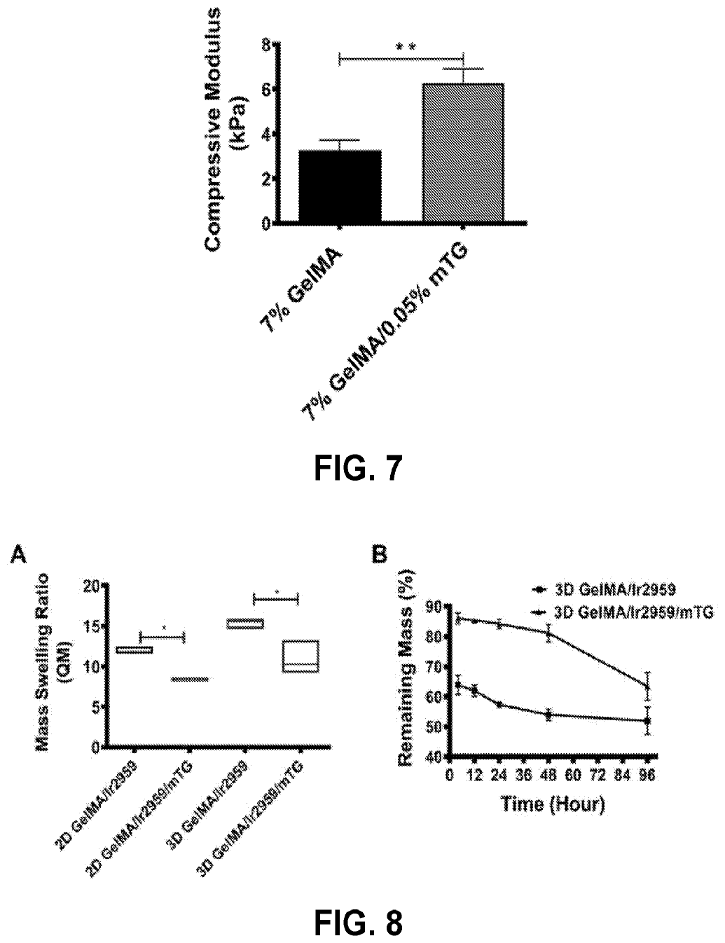

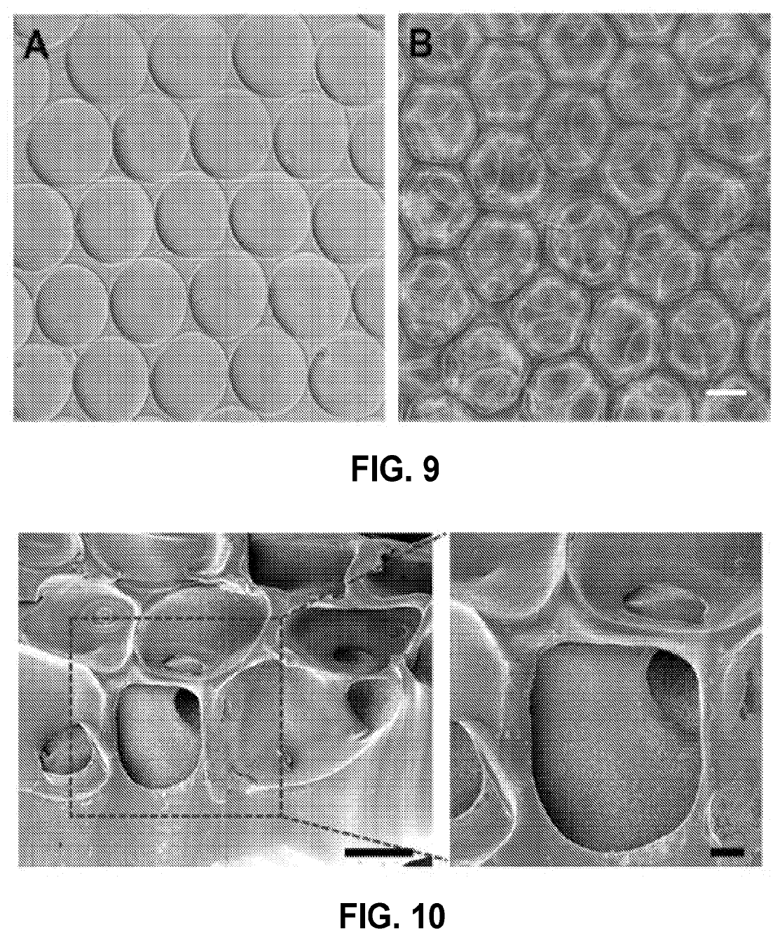

Further, 7% GelMA was chosen since it allowed sufficient stability of the resulting sac-like structures while having a stiffness close to the normal lung tissue; in various embodiments GelMA may be used in a range of 1%-25% to vary the stiffness of the hydrogel. GelMA was used as the model ECM to fabricate the scaffold due to its biocompatibility, photo-crosslinkability, availability, and cost-effectiveness. Importantly, GelMA possesses some of the essential properties of ECM such as presence of arginine-glycine-aspartic acid sequences, which promote cell attachment, and matrix metalloproteinase-responsive peptide motifs, thus allowing cells to proliferate and spread in/on GelMA-based scaffolds. A range of modulus values has been reported for the human lung tissue depending on the anatomical compartments of the lung and the mechanical testing techniques used, from ˜1.4 to 7.2 kPa. The compressive modulus of our GelMA hydrogels (double cross-linked; first with Irgacure 2959 and then with microbial transglutaminase [mTG]) was found to be 6.23±0.64 kPa (FIG. 7), which is within the range of the mechanical strengths reported for the native lung tissue. In addition, the swelling ratios of the GelMA hydrogels (double cross-linked) were significantly smaller (˜8 and ˜10% for 2D and 3D GelMA hydrogels, respectively) than those photo-crosslinked with Irgacure 2959 alone (˜12 and ˜15% for 2D and 3D GelMA hydrogels, respectively), as shown in FIG. 8A. Thus, we first photo-crosslinked our GelMA constructs using Irgacure 2959 and then further chemically cross-linked them using mTG to increase their stability as we have shown also in the degradation profiles in FIG. 8B. We have further used collagen methacryloyl to fabricate an inverse opal structure, demonstrating the feasibility of our approach to engineer the alveolar lung model using different hydrogel biomaterials (FIG. 9).

The structure (all based on GelMA below) was further characterized by scanning electron microscopy, as illustrated in FIG. 3A, subpanel ii, Inset, and FIG. 10, in which an interconnecting window between two adjacent sacs could be observed. The fluorescence confocal images of the fluorescein isothiocyanate (FITC)-conjugated GelMA before and after alginate microbead removal further verified their structure (FIG. 3A, subpanel iii).

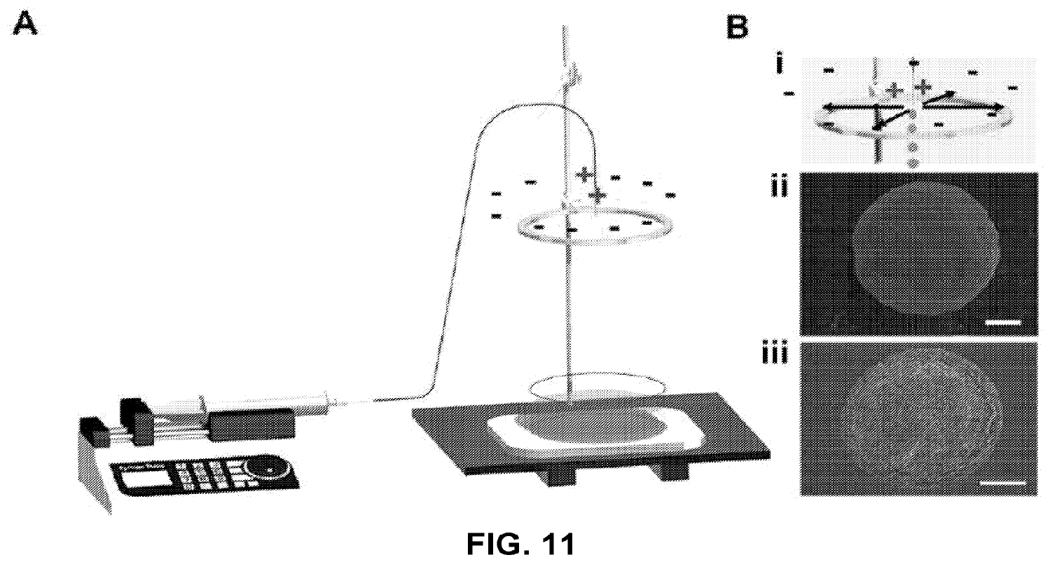

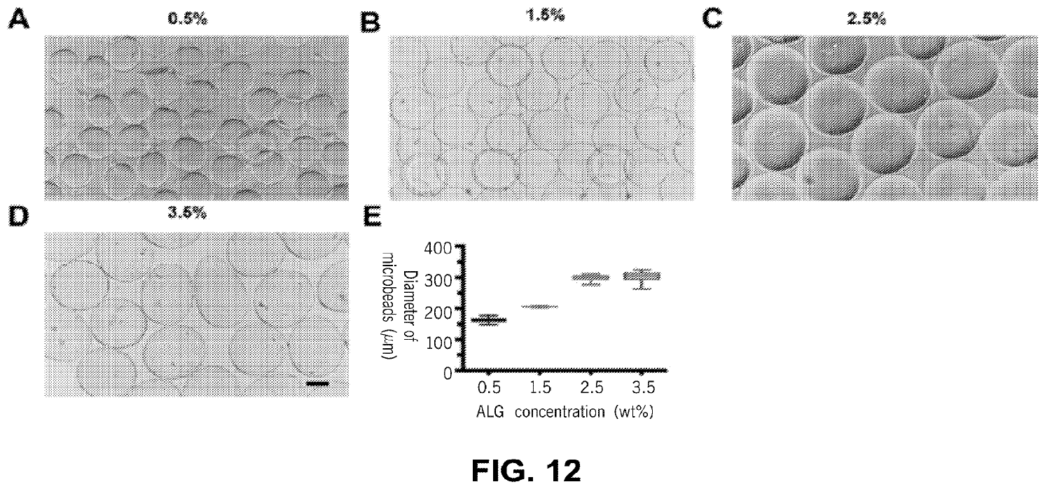

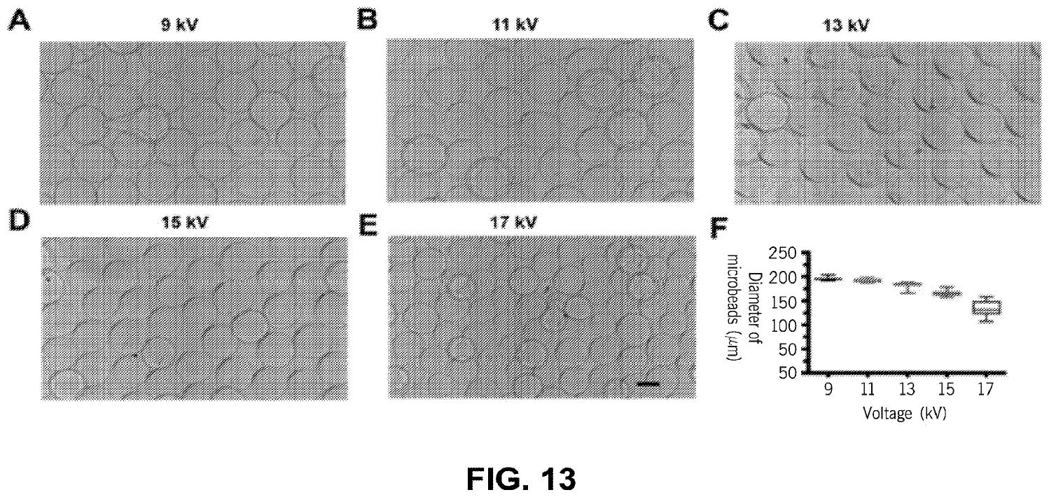

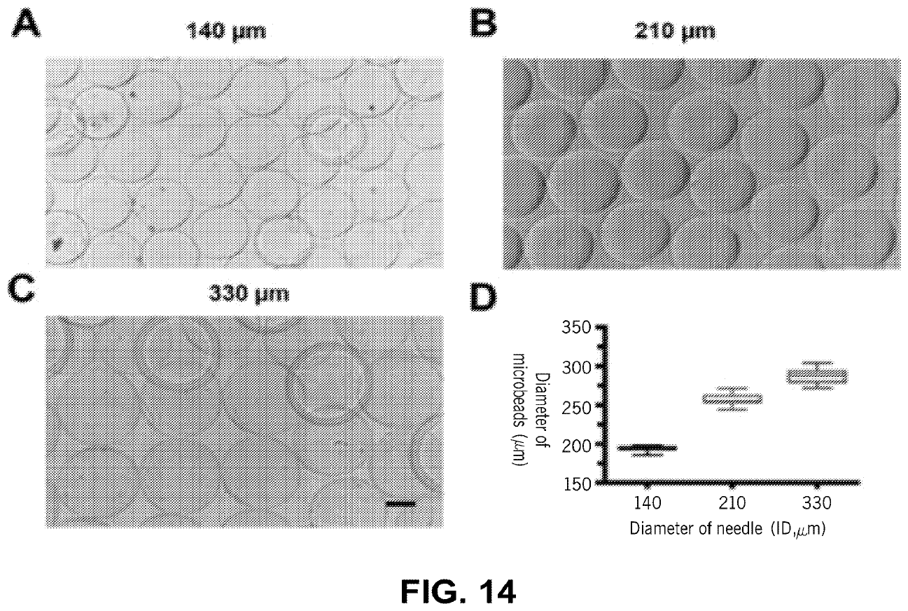

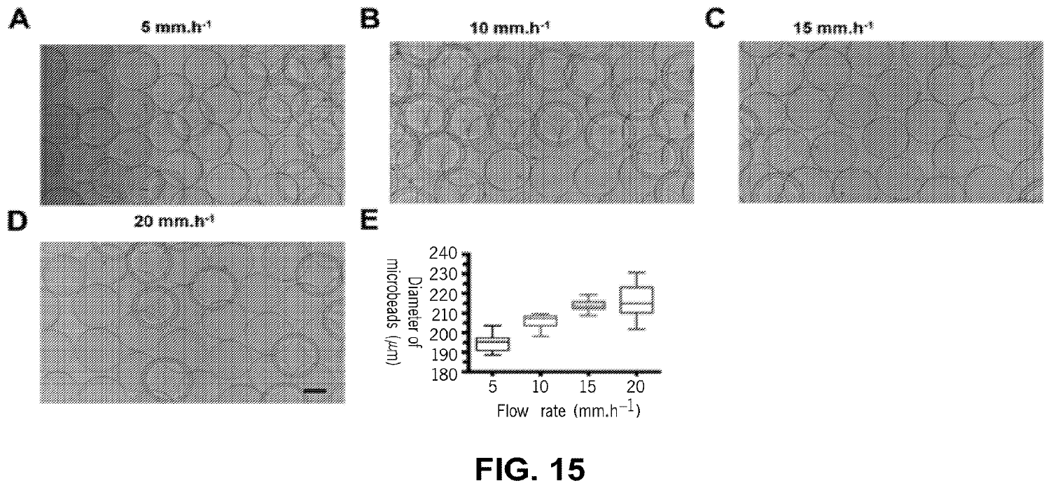

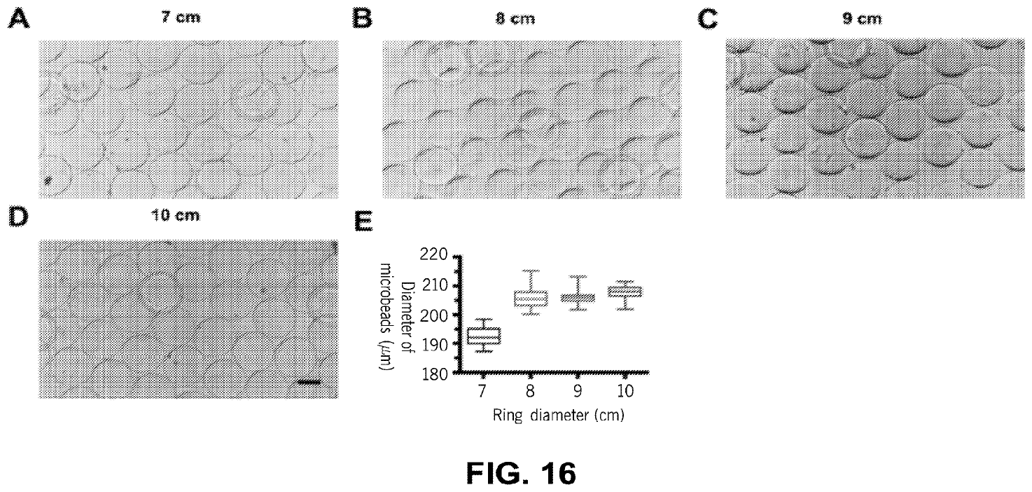

The alginate microbeads were prepared by electrospraying the alginate solution into a CaCl₂) solution for immediate physical cross-linking (FIG. 11). The influence of each parameter, including alginate concentration, voltage, needle diameter, flow rate, and ring diameter, on the formation of the microbeads was carefully studied (FIG. 12-FIG. 16). While a wide range of diameters could be conveniently achieved, uniform alginate microbeads measuring ˜200 μm (201±12 μm) were selected for constructing the alveolar lung model since the average size of each alveolus is ˜200 μm in diameter in the native lung. Approximately 7,050 microbeads were packed into three layers, resulting in a roughly 546.4-μm-thick inverse opal structure. Such an inverse opal structure represented the miniature alveolar lung model that featured the sac-like pores and the interconnecting windows between the sacs, which resembles the native human counterpart in both architecture and dimensions, meaning that the two would also share the same unit surface area. The dimension of the overall 3D GelMA scaffold was 8×10×3 mm³ that included a layer of bulk GelMA base below the 546.4-μm-thick inverse opal GelMA layer for improved stability.

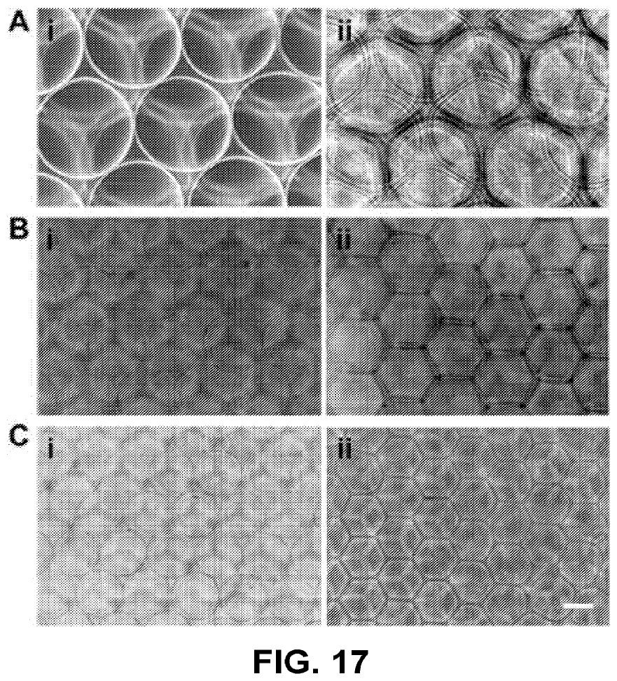

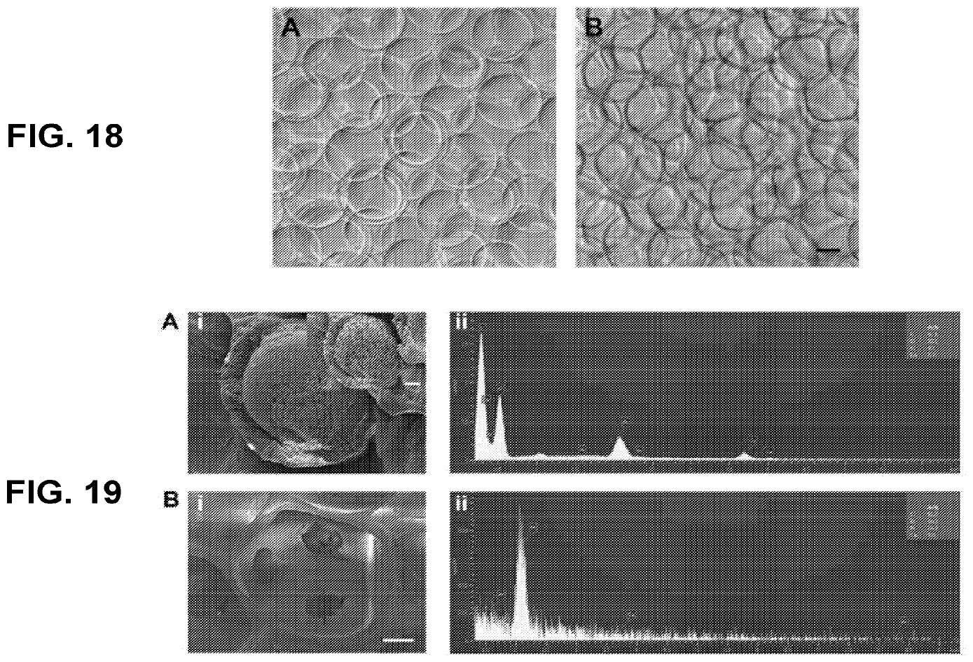

It should be noted that, although the human alveoli are not necessarily homogeneous in size, in our model we pursued the use of only uniform alginate microbeads as the sacrificial templates to avoid unwanted interferences caused by the heterogeneity in the alveolar sizes. Indeed, we have previously demonstrated that the use of porous scaffolds with uniform pores is experimentally superior to those with random pores even if the average pore size falls in the same range. In fact, we were also able to fabricate the 3D inverse opal GelMA structures with different yet uniform pore sizes using monodispersed alginate microbeads of different diameters. The microbeads with sizes of 325.9±10.5, 199.6±1.1, and 156.3±2.7 μm were used separately to fabricate 3D GelMA structures with corresponding pore sizes (FIG. 17). Likewise, we could also fabricate a 3D GelMA structure with heterogeneous pore sizes by using randomly mixed microbeads with three diameters (˜200, ˜250, and ˜300 μm) as the template (FIG. 18). The alginate microbeads could be rapidly removed by treating with the 0.01 M EDTA solution. The complete removal of the alginate microbeads from the scaffold was confirmed by both scanning electron microscopy imaging and X-ray energy dispersive spectroscopy (EDS) mapping, shown in FIG. 19. In particular, EDS mapping profiles suggested that while 1.3% of Ca²⁺ was detected in the sample before removal of the microbeads, no Ca²⁺ (0%) was measured at all after the removal. Thus, removal of both Ca²⁺ and the alginate microbeads was complete.

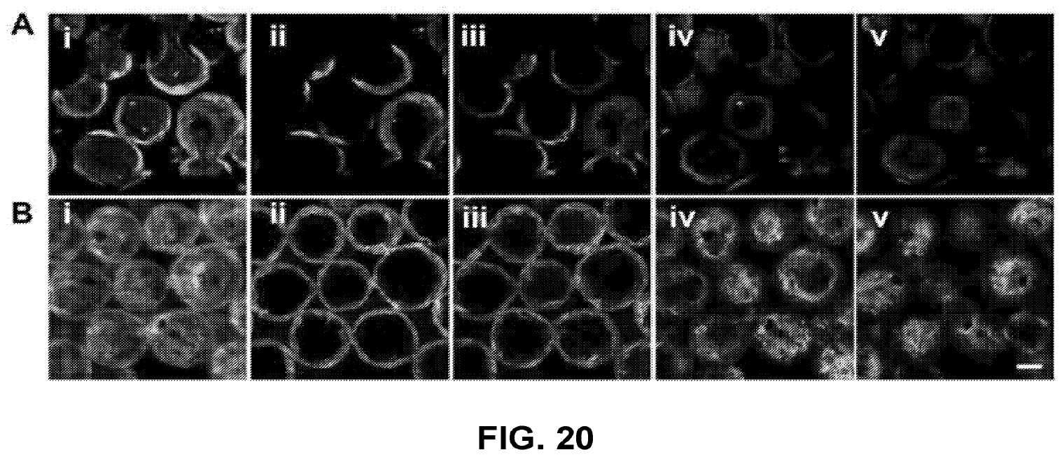

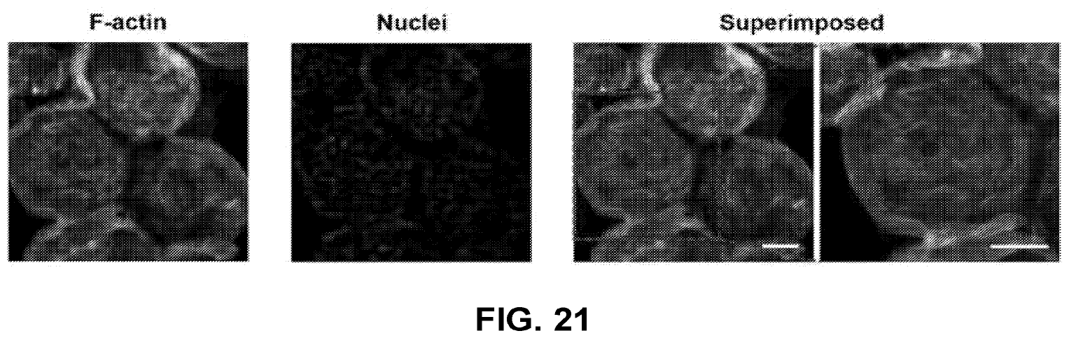

To model the alveolar epithelium, we populated hAECs on the surfaces of the sacs within the GelMA inverse opal structures. Fluorescence confocal images revealed that the hAECs were almost rounded and crowded at day 1 post-seeding, where they were able to gradually spread and proliferate during the following days, eventually covering the full surface areas of the alveoli-like sacs (FIG. 3B, subpanel i; FIG. 3C; FIG. 20; and FIG. 21). The viability of the hAECs on the surfaces of the pores in the porous GelMA structures was observed to exceed 95% (FIG. 3B). The proliferation of the cells was steady during the first 10 d of culture and slightly slowed down afterward (FIG. B, subpanel ii), possibly due to the contact inhibition upon confluency of the cells on the surfaces of the pores. The cell viability, proliferation, and spreading at the different depths within a single layer of the sacs of the inverse opal structure are shown in FIG. 20. Indeed, fluorescence confocal images suggested that these culture conditions resulted in the formation of a confluent alveolar epithelium (FIG. 3, FIG. 20, FIG. 21). Significantly, the hAECs were linked by a continuous presence of tight junctions as indicated by zonula occludens (ZO)-1 staining (FIG. 3D). Furthermore, hematoxylin and eosin (H&E) staining provided an overview of the structure of the GelMA hydrogel and the cell distributions, clearly validating the emulation of the alveoli-like, interconnected sacs and the continuous monolayer of hAECs on the pore surfaces (FIG. 3E).

Comparisons between 2D and 3D Cultures of the Human Alveolar Epithelium

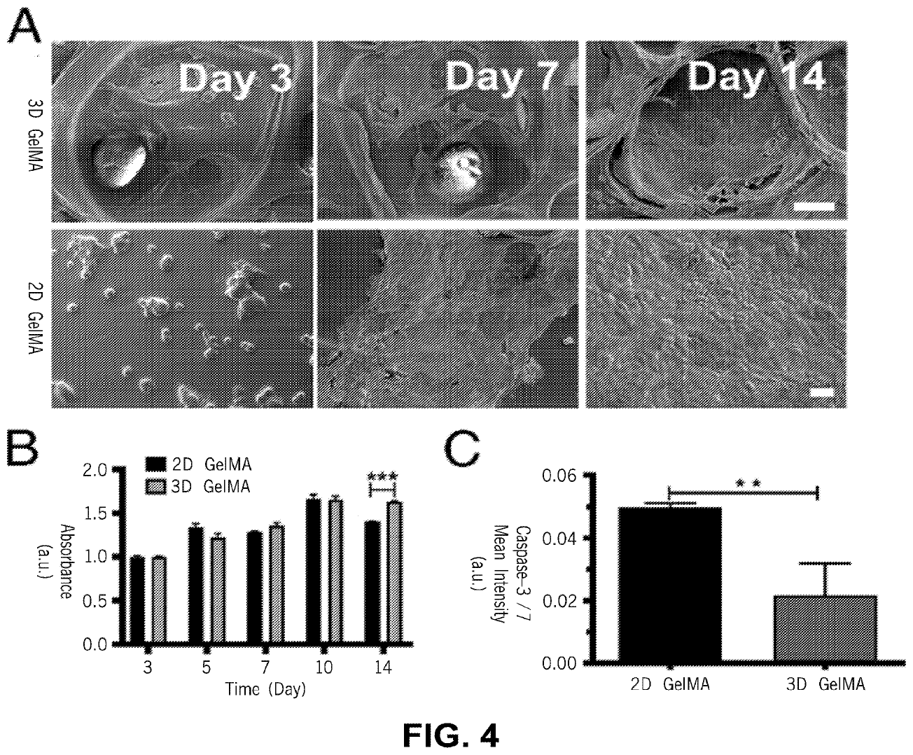

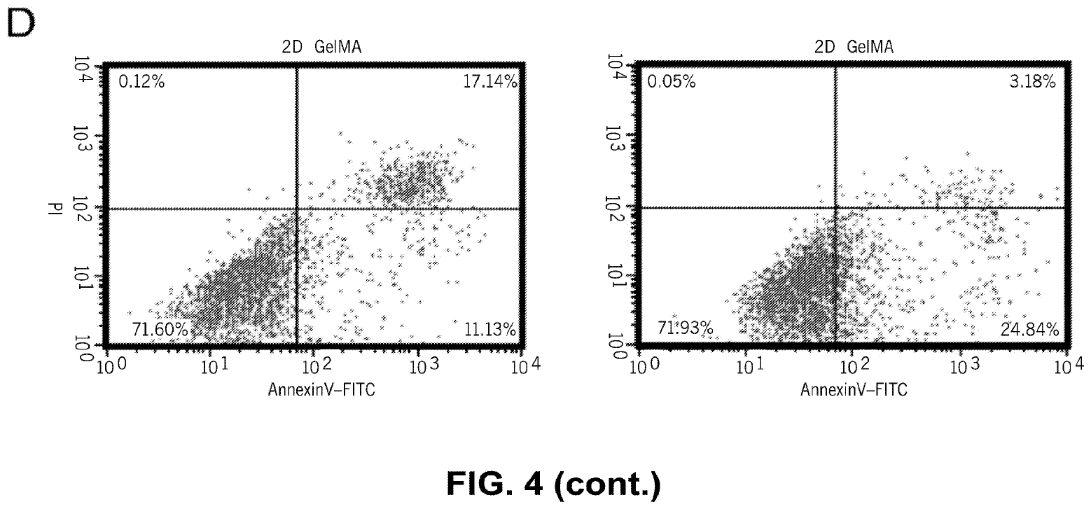

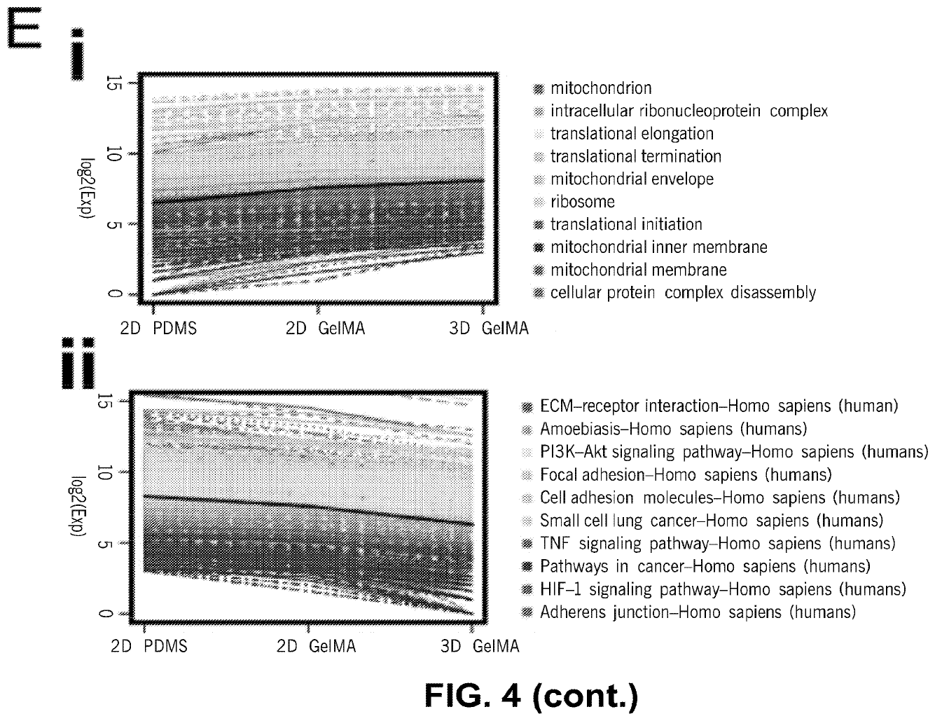

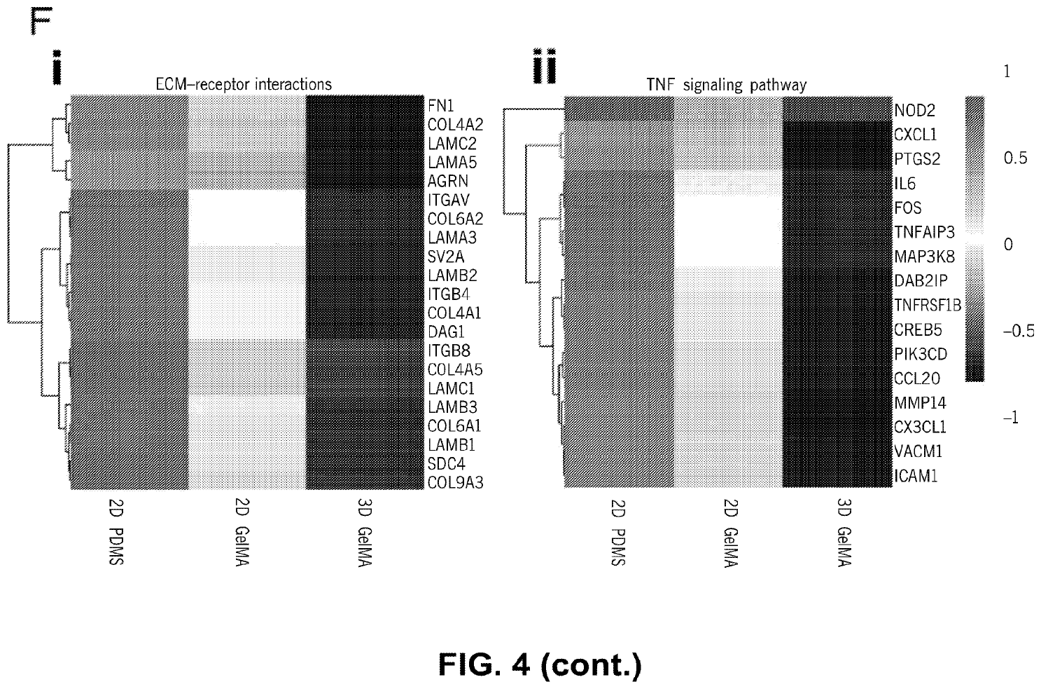

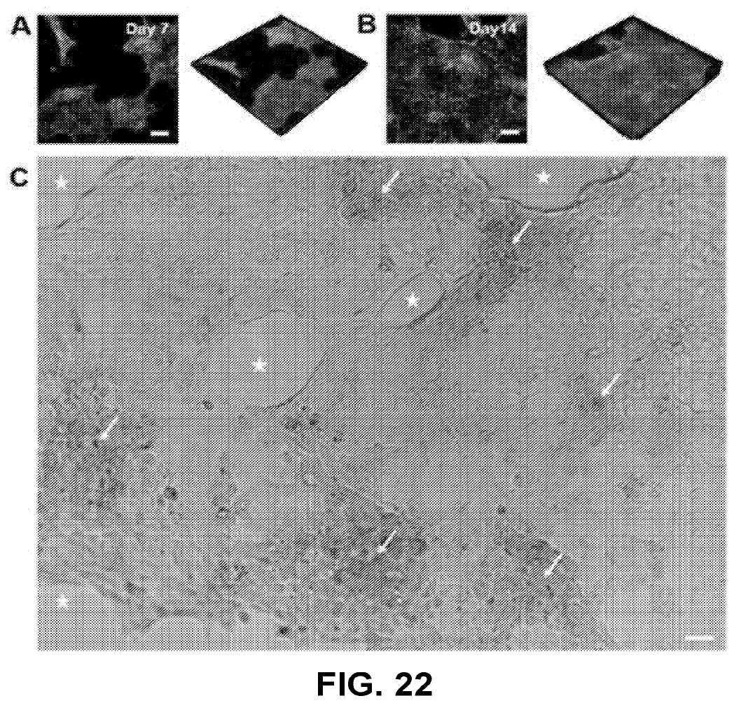

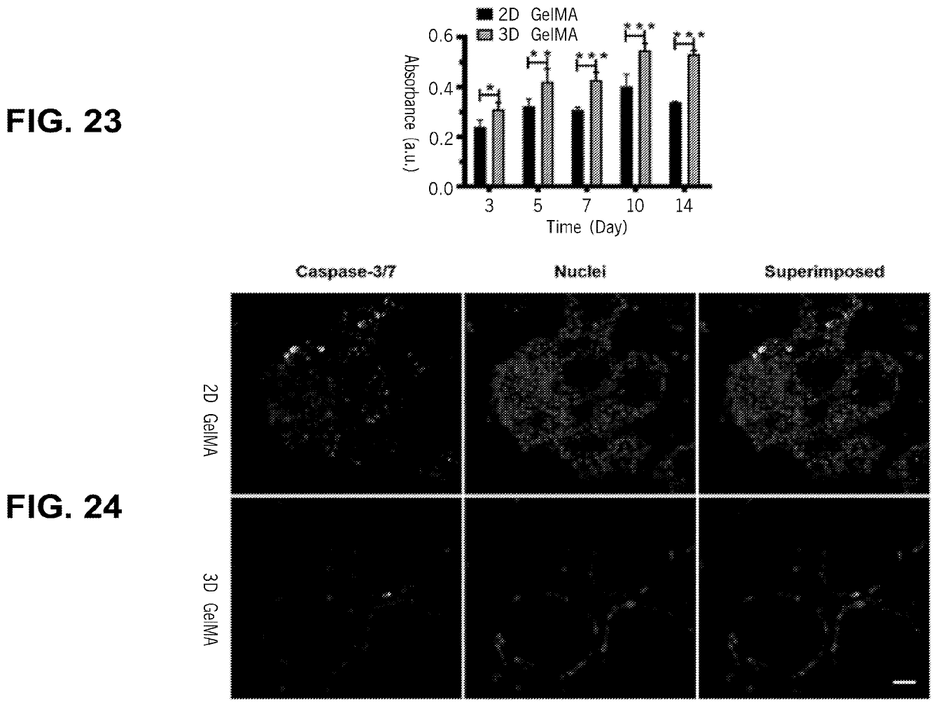

Reconstitution of the 3D structures and microenvironments of the tissues is vital for realizing their functions and modeling pathophysiology in vitro. To investigate the advantages of our 3D GelMA inverse opal structures in the recapitulation of the pulmonary alveoli, the behaviors of hAECs were carefully compared with those cultured on 2D GelMA surfaces. The morphologies of the hAECs were further studied by capturing scanning electron microscopy images (FIG. 4A) of the cells cultured on both the 3D GelMA structures (FIG. 4A, Upper) and the 2D GelMA surfaces (FIG. 4A, Lower) at days 3, 7, and 14. Comparing with the slightly crowded and oftentimes locally aggregated hAECs on the 2D GelMA surfaces during the culture, the hAECs in the 3D GelMA structures appeared to be flatter, where the formation of a smooth, confluent epithelium by well-spread cells was apparent at day 14. The microscopic images of hAECs cultured on the 2D GelMA surfaces are shown in FIG. 22. Moreover, the apparent proliferation rate of the hAECs in the 3D GelMA structures was significantly higher than that of the cells on the 2D surfaces (FIG. 23). Nevertheless, when normalized (FIG. 4B), there were no significant differences between the two configurations anymore during the culture period of 10 d. The hAECs in the 3D GelMA structures became higher in numbers than those on the 2D surfaces at day 14, most likely due to the presence of the larger total surface area within the 3D GelMA structure as compared with the 2D surface, at ˜2 cm² in the former vs. 0.8 cm² for the latter despite that they share the same planar size of 8×10 mm. Most importantly, the 3D GelMA scaffolds provided an optimal geometry and microarchitecture for cell spreading and proliferation compared with the planar surfaces, which possibly promoted the cellular process such as adhesion, proliferation, and migration. These observations indicated that the 3D culture provided an optimal geometry for cell spreading and proliferation compared with the planar culture when both substrate materials were the same GelMA with similar local mechanical properties, both close to that of the normal human lung tissue.

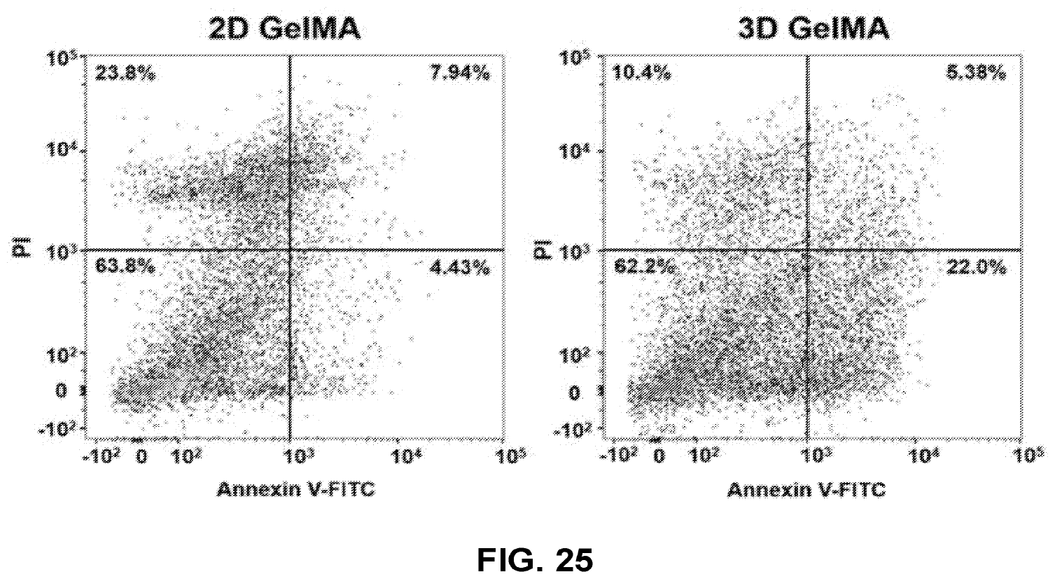

Apoptosis is a fundamental cellular process that is necessary for the maintenance of tissue homeostasis in adult organisms. The apoptotic activities of the cells were analyzed via caspase-3/7 staining of hAECs and flow cytometry analyses of annexin V-FITC- and propidium iodide (PI)-stained hAECs both on 2D GelMA surfaces and in 3D GelMA structures. Interestingly, it was found that the mean fluorescence intensity of caspase-3/7 was higher for the cells grown on the 2D GelMA surfaces compared with those in the 3D GelMA structures (FIG. 4C and FIG. 24). Similarly, the flow cytometry analyses of annexin V-FITC- and PI-stained hAECs at day 14 and day 22 showed that the overall proportion of late apoptotic/necrotic hAECs on the 2D GelMA surfaces was increased over the culture period compared with those in the 3D GelMA structures (FIG. 4D and FIG. 25). While the proportions of early apoptotic cells were higher in 3D structures, the proportions of the late apoptotic/necrotic cells were higher on the 2D GelMA surfaces, at both day 14 (FIG. 4D) and day 22 (FIG. 25). It is therefore hypothesized that our unique 3D GelMA inverse opal structure resembling that of the human alveoli could possibly provide the cells with an in vivo-like microenvironment, which promoted optimal cell-ECM and cell-cell interactions.

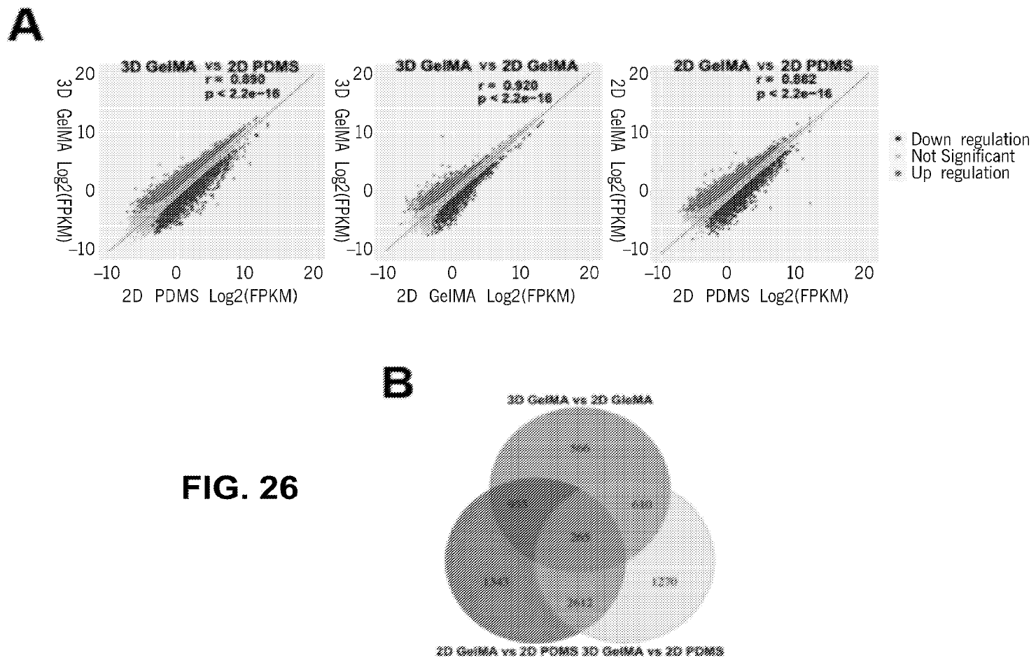

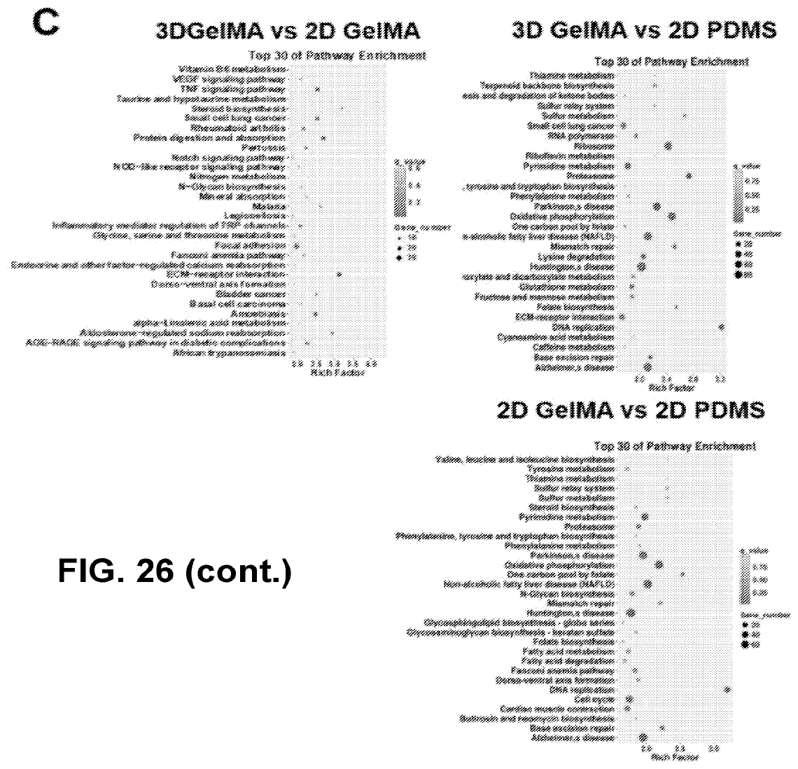

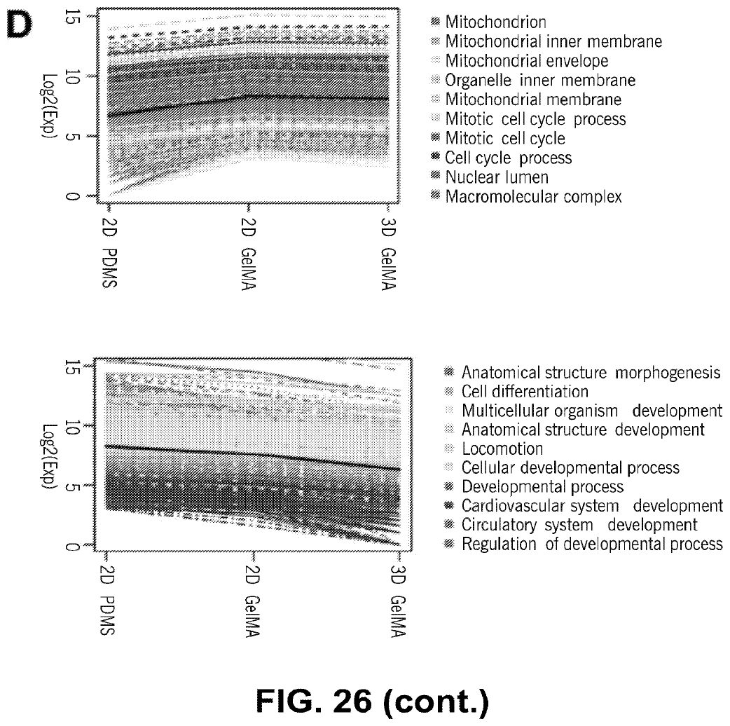

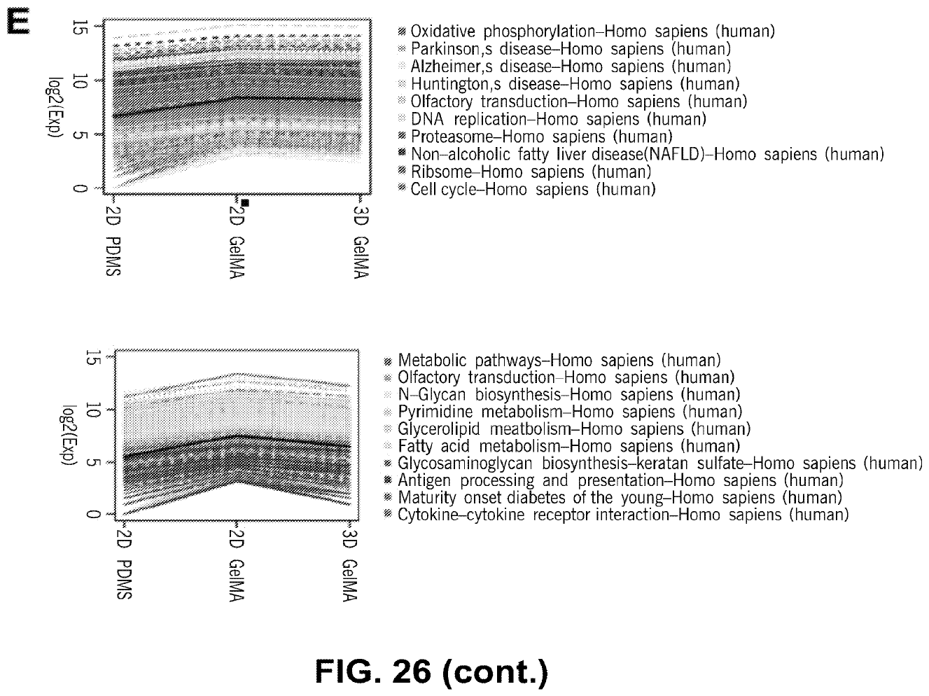

To explore the differentially expressed genes to detect biological changes in hAECs cultured in 2D and 3D, we performed additional transcriptome profiling through gene microarray analyses. Other than the 2D GelMA surfaces, we further included another control group based on the 2D PDMS surfaces (a commonly used substrate in various organ-on-chip models, including those of the lung) to compare with the 3D GelMA structure-based hAEC culture. Scatterplots and the Venn diagram (FIG. 26A, 26B) were generated for the three comparisons (i.e., 3D GelMA vs. 2D PDMS, 3D GelMA vs. 2D GelMA, and 2D GelMA vs. 2D PDMS). We calculated the normalized expression values (fragments per kilobase per million mapped reads) of every gene analyzed in the three groups, and those values with greater than twofold increase or decrease with a false discovery rate of less than 0.05 (q value <0.05) were considered as the differentially expressed genes.

Interestingly, the 3D GelMA vs. 2D PDMS comparison demonstrated that there were 4,757 differentially expressed genes (2,646 up-regulated and 2,111 down-regulated); for 3D GelMA vs. 2D GelMA, there were 2,396 differentially expressed genes (1,352 up-regulated and 1,044 down-regulated), while for 2D GelMA vs. 2D PDMS, there were 5,175 differentially expressed genes (2,553 up-regulated and 2,622 down-regulated) (FIG. 26B). To elucidate the biological processes on the different culture substrates, functional gene ontology (GO) and Kyoto Encyclopedia of Genes and Genomes (KEGG) pathway enrichment analyses were further performed (FIG. 26C). Through K-means clustering analysis of the differentially expressed genes in the three groups, heat maps relating to GO and KEGG enrichment analyses of clusters showed similar gene expression trends (FIG. 4E, FIG. 26D, FIG. 26E).

We have specifically focused on some key functional categories to better understand the major differences for the hAECs cultured on 2D PDMS, 2D GelMA, or 3D GelMA, exhibited as the GO enrichment analysis in FIG. 4E, subpanel i, and KEGG enrichment analysis in FIG. 4E, subpanel ii. The genes associated with ECM-receptor interactions, focal adhesion, tumor necrosis factor (TNF) signaling pathway, and small-cell lung cancer in the clusters had a monotonically decreasing trend from 2D PDMS and 2D GelMA to 3D GelMA cultures, while oxidative phosphorylation in the cluster showed an increasing trend (FIG. 26). Similarly, biological processes regarding mitochondrial biogenesis also possessed an increasing trend in the order of 2D PDMS. 2D GelMA, and 3D GelMA cultures. Mitochondria provide the cells with adenosine triphosphate through the oxidative phosphorylation activity, and up-regulation of these pathways could provide more energy to help maintain the functionality of the cells. Specifically, heat maps were produced to compare differentially expressed genes involved in ECM-receptor interactions and the TNF signaling pathway. Integrin-β4 (ITGB4), which is one of the transmembrane receptors for ECM proteins, can form a stable attachment to the basal membrane in polarized epithelial cells through the formation of hemidesmosomes and also has a significant impact on signaling events that stimulate migration and invasion. It was observed that the hAECs expressed more ITGB4 and interleukin-6 (IL-6) in 2D conditions (both GelMA and PDMS) than in 3D GelMA (FIG. 4F), possibly indicating a response to the more unnatural 2D microenvironments. Focal adhesions are key transducers of outside-in signaling that enable cells to sense cues from the ECM and generate forces. In 2D cultures, especially for PDMS, up-regulation of focal adhesion genes and ECM-receptor interaction genes might be caused by the stiff and pathologically activated surfaces. Prior studies further reported that TNF-induced death signaling induced alveolar epithelial dysfunction in acute lung injury and also, the increase of IL-6 expression in lungs with diffuse alveolar damage. Collectively, these analyses hinted that our 3D GelMA inverse opal structure provided better capabilities to maintain and promote the functions of hAECs in a more in vivo-like manner compared with 2D models, particularly those based on the planar PDMS surfaces.

Construction of a breathing human alveolar lung-on-a-chip model

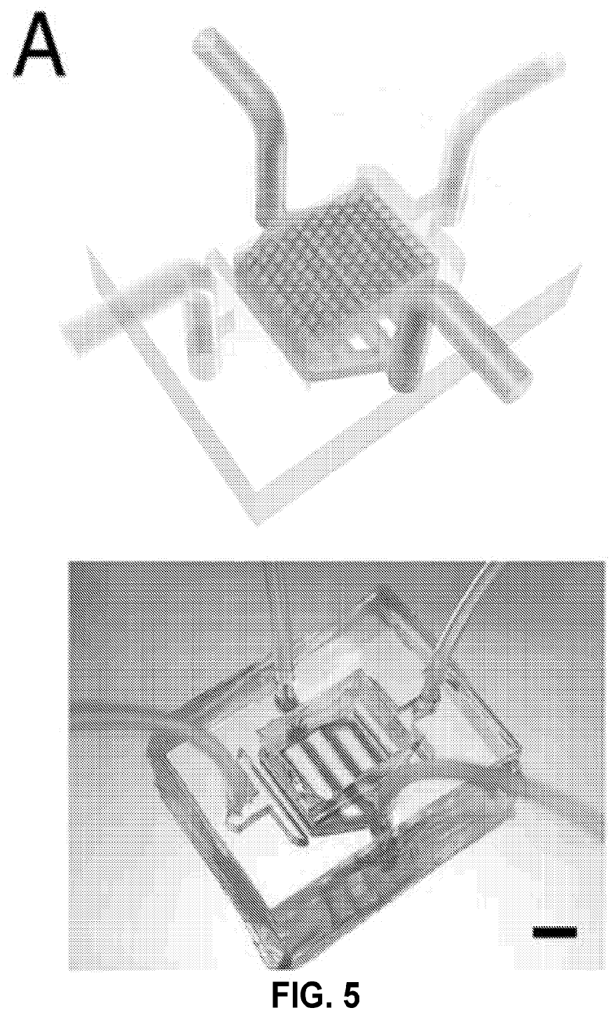

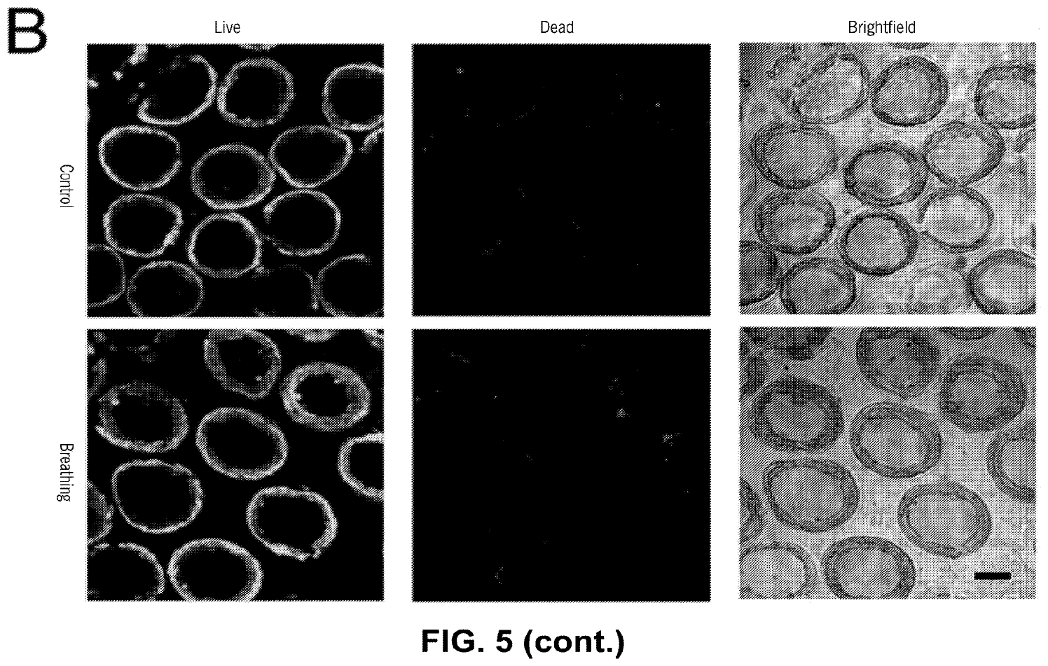

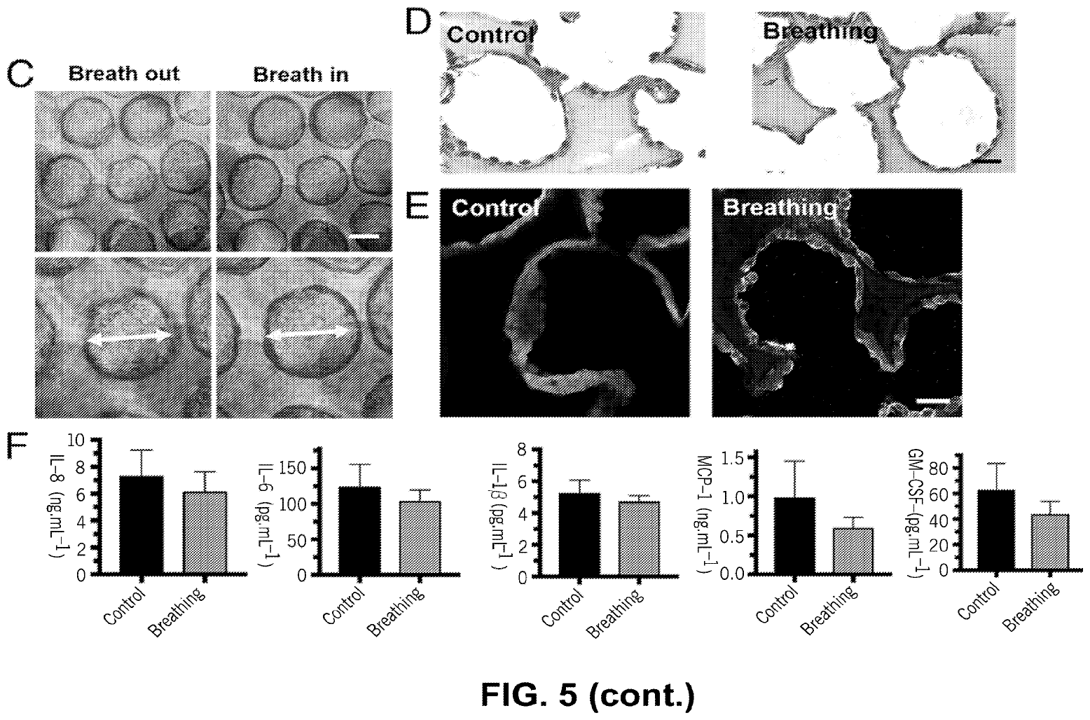

A schematic diagram of the alveolar lung-on-a-chip (Upper) and a photograph of an actual device are shown in FIG. 5A. The fabrication process of the chip is schemed in FIG. 27. The chip included the 3D porous GelMA inverse opal scaffold (red part in the schematic in the upper portion of FIG. 5A) bonded to a PDMS chip. The PDMS support was designed to provide the 3D hydrogel with the medium supply and cyclic mechanical actuations. The medium supply was realized by slowly infusing the medium through the open channels at the bottom of the PDMS chip. The cyclic mechanical movements were applied to the 3D hydrogel via applying the programmed negative pressures to the two side chambers at the frequency of 0.2 Hz (the normal breathing frequency of humans).

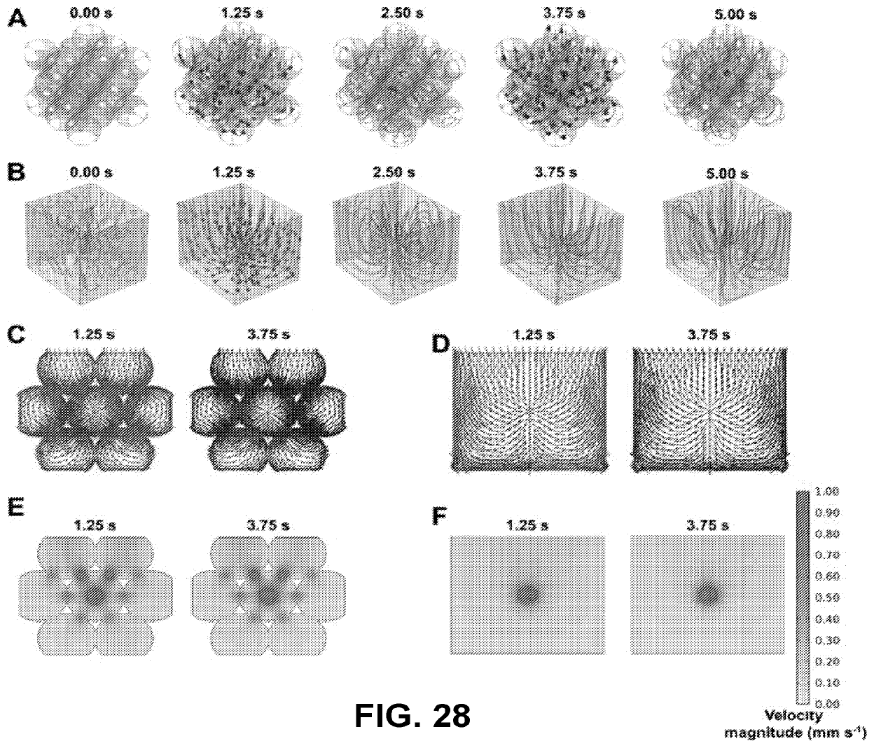

The mechanical movements of the chip allowed the airflow in and out of the embedded 3D GelMA structure. There are some reports that have focused on building and simulating the airflow in the artificial alveoli; however, only a few other groups have successfully reconstituted the 3D structures of the human alveolar sacs. Herein, we demonstrate the airflow in and out of the alveoli-like structure in our inverse opal alveolar lung model during a breathing cycle, as shown in the simulation results in FIGS. 28A, 28C, and 28E. During the “breathing” of the chip, the air gradually flowed into and then out of the inverse opal sacs spontaneously due to the volume change-induced pressure variation with velocity values and Reynolds numbers (0.71 to 1.70), which are very similar to those found in the real human alveoli. In addition, direction of the airflow changed according to the structural framework within the inverse opal alveolar lung model, especially in correspondence at the “alveolus-alveolus” interfaces. On the contrary, in the control 2D model, the airflow followed nonphysiological straighter paths without showing rapid changes of directions (FIGS. 28B, 28D, and 28F).

After seeding the hAECs in the 3D GelMA inverse opal structures in the chips, the cells were cultured for 14 d. Live/dead images revealed that the cell viability remained high after 14 d of static culture in the chips (FIG. 5B, control). When the cyclic stretch was applied, the hAECs grown on the surfaces of the pores could “breathe” along with the cyclic mechanical movements that stretched the GelMA structure, where the average size of alveoli was observed to expand by ˜8% from the state of “breath out” to “breath in” (FIG. 5C). This value is within the 5% to 15% physiological range of strain experienced by the alveoli in the human lung. It was, in fact, convenient for us to control the strains or breathing frequencies on the chip as needed by varying the size of the two side chambers or the frequency of the negative pressure applied. Our chip-based model functioned well under a range of strain levels of 5, 10, and 15%, as well as breathing frequencies of 0.1, 0.2, 0.4, and 0.8 Hz.

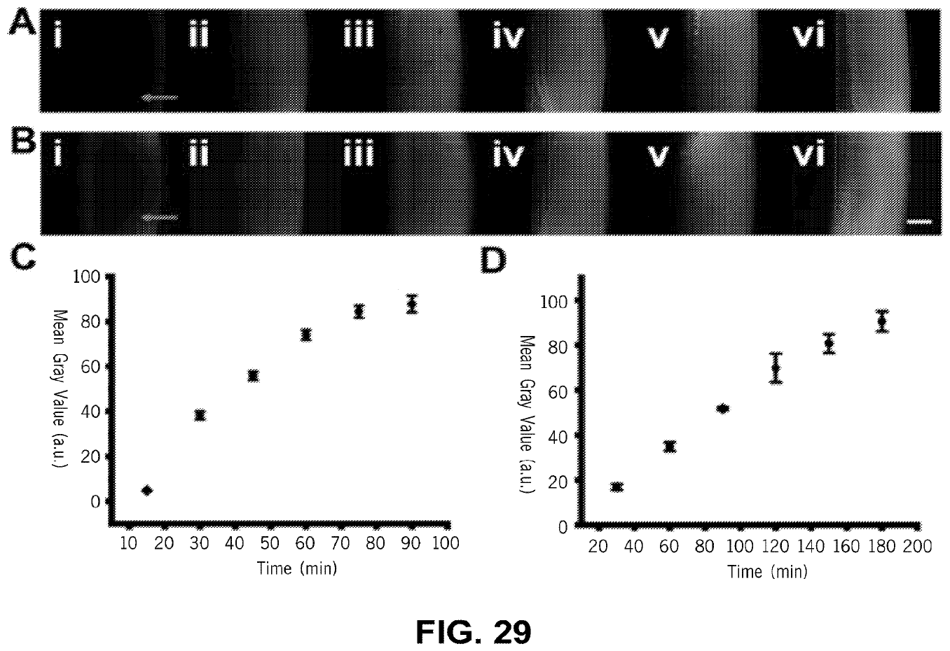

An ALI was also created for the hAECs in the chip by removal of the medium from the sacs and supplying the medium through the parallel channels underneath the GelMA structure at the bottom of the chip. The relatively low thickness (<3 mm) of the GelMA hydrogel and its nano-/microscale porosities were believed to enable efficient medium transport to support cellular functions. To confirm the sufficient supply of nutrients within the top layer of the 3D GelMA inverse opal structure, we assessed the diffusion of both small molecules (FITC) as well as larger molecules (FITC-dextran, M_{w}=10 kDa). It was found that the diffusion of both types of molecules across the entire GelMA structure occurred within relatively short periods (FIG. 29). Since the diffusion is a continuous process, this observation potentially suggested good transport of various components within the culture medium through the GelMA structure.

Indeed, the hAECs cultured within the alveolar lung-on-a-chip devices remained viable after the incorporation of the ALI and the application of the breathing events for 48 h (FIG. 5B, breathing), while the formed alveoli-like epithelial monolayer within the GelMA inverse opal structure could be completely preserved as revealed in the H&E-stained sections (FIG. 5D). Moreover, the presence of cyclic mechanical strains seemed to have promoted the formation of tight junctions, indicated by ZO-1 staining (FIG. 5E). The levels of secreted cytokines including IL-8, IL-6, IL-1B, monocyte chemoattractant protein-1 (MCP-1), and granulocyte-macrophage colony-stimulating factor (GM-CSF) were further profiled; they were all slightly reduced upon breathing stimulation, yet no significant differences could be found (FIG. 5F).

### Analysis of Cigarette Smoking Effects on the Alveolar Lung-on-a-Chip Model

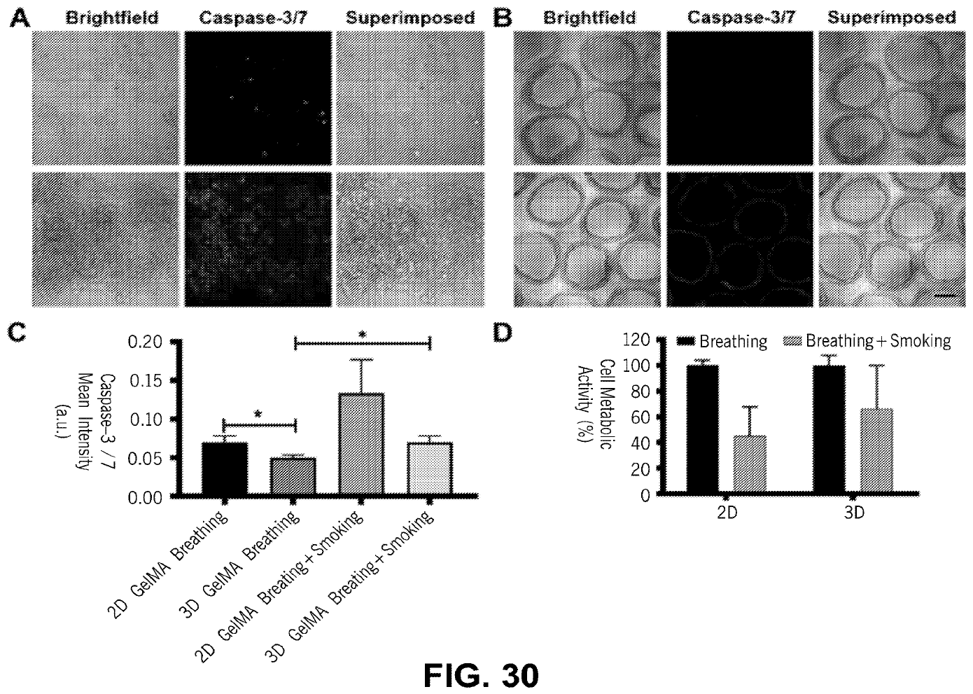

Cigarette smoking is one of the major factors leading to the development of COPD, currently the third leading cause of chronic morbidity and mortality worldwide, as well as resulting in lung cancer as the most common lethal neoplasm. Since the alveolar epithelium is the barrier between the inhaled air and the underlying components, the establishment of a smoking alveolar lung-on-a-chip model to examine the distal lung injury and study the effects of smoking on the alveolar epithelium becomes important. While a smoking lung-on-a-chip platform was previously demonstrated using airway epithelial cells on a synthetic polyethylene terephthalate membrane, no model currently exists for the distal lung (i.e., the alveoli). Our smoking alveolar lung-on-a-chip model included a smoking device and an additional compartment on top of the breathing alveolar lung-on-a-chip platform to mimic the airway-alveoli transport route (FIG. 6A). After smoking ˜10 cigarettes and further culturing for 24 h, dead cells within our alveoli model showed an increase (FIG. 6B), and the viability of hAECs was significantly decreased compared with normal breathing and control groups (FIG. 6C). The apoptotic activities of the cells before and after the application of the cigarette smoke were analyzed via caspase-3/7 staining of hAECs on/in both 2D and 3D scaffolds (FIG. 30). Consistent with the cultures without the chips (FIG. 4 and FIG. 24), hAECs on 2D GelMA in the chip also showed a higher apoptotic tendency (FIG. 30A) as compared with those in 3D GelMA structures (FIG. 29B). The smoking further resulted in the increase of apoptosis of hAECs in both configurations (FIG. 30A-30C). This observation was, in addition, reflected in cellular activity assay (FIG. 30D).

In addition, the tight junctions between the hAECs were partially damaged after smoking (FIG. 6D). The oxidants in cigarette smoke can induce cellular injuries, where 4-hydroxy-2-nonenal (4-hydroxynonenal [4-HNE]) is a major product of lipid peroxidation and a marker of oxidative stress. 4-HNE can react with DNAs and proteins to produce various adducts that can lead to cell apoptosis, and the 4-HNE expression level in the alveolar epithelium is negatively correlated with pulmonary functions and contributes to the pathogenesis of various pathological manifestations such as COPD. Our results indicated an elevation in the intensity of nuclear 4-HNE staining post-smoking (FIG. 6E), and such a high expression of 4-HNE may further induce downstream cellular damage. As a result, the soluble cytokine levels of IL-8, IL-6, IL-1B, MCP-1, and GM-CSF after smoking slightly declined compared with the normal breathing group, suggesting that the functions of hAECs to inhibit proinflammatory responses were compromised when they were exposed to cigarette smoke, consistent with literature.

Analysis of SARS-COV-2 pseudoviral infection and treatment efficacies on the alveolar lung-on-a-chip model

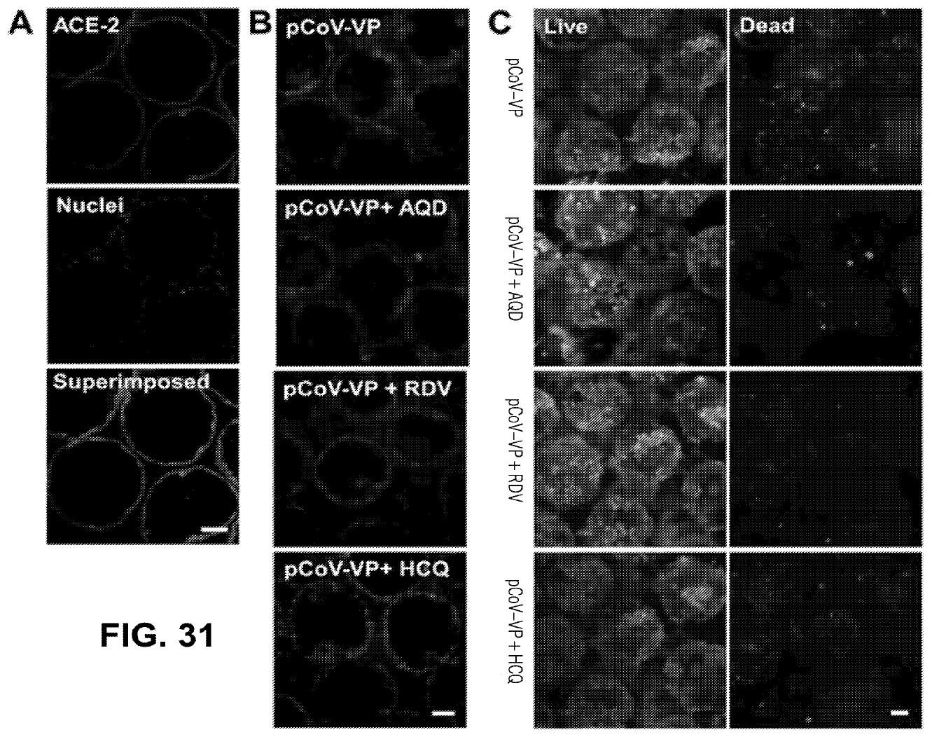

COVID-19 caused by SARS-COV-2 has led to a global public health crisis and is associated with high mortality, mostly caused by acute respiratory distress syndrome. As the lungs are the first body organ affected by COVID-19, we further assessed the applicability of our alveolar lung-on-a-chip to investigate SARS-COV-2 pseudoviral infection. In addition, the antiviral efficacies of two clinically approved antiviral drugs, remdesivir and hydroxychloroquine, as well as an antimalarial drug, amodiaquine, were evaluated. The latter has also been given to individuals infected with SARS-COV-2. Since it has been well recognized that SARS-COV-2 uses angiotensin converting enzyme 2 (ACE2) as a receptor for cellular entry, prior to SARS-CoV-2 pseudoviral particle (pCoV-VP) infection on the alveolar lung-on-a-chip, we first detected the expression of ACE2 receptors by hAECs grown on the surfaces of the alveolar sacs. The hAECs showed a higher level of expression of ACE2 receptors as shown in FIG. 6G and FIG. 31A. The chips were then inoculated with pCoV-VPs at multiplicity of infection (MOI) of 0.5 for 48 h to identify the virus susceptibility in vitro in the presence and absence of antiviral drugs. At 48 h postinfection, the infection of hAECs with mcherry-labeled pCoV-VPs was observed under a fluorescence microscope (FIG. 31B), and cytopathic effect of pCoV-VPs was assessed by live/dead assay (FIG. 6H and FIG. 31C) and 3-(4,5-dimethylthiazol-2-yl)-5-(3-carboxymethoxyphenyl)-2-(4-sulfophenyl)-2H-tetrazolium (MTS) assay (FIG. 6I). The results showed that the cytopathic effects of pCoV-VPs were decreased by approximately 21.4, 30.0, and 11.3% at 5 μM amodiaquine, 10 μM remdesivir, and 40 μM hydroxychloroquine, respectively (FIG. 6I). Thus, these drugs significantly inhibited the infection-induced hAEC death by the SARS-COV-2 pseudotyped viral particles expressing the SARS-Cov-2 spike proteins in our alveolar lung-on-a-chip.

This disclosure provides examples of embodiments of an apparatus, system, and/or methods for recreating the architecturally relevant alveolar lung-on-a-chip model and demonstrated its structural and functional features. We have successfully reconstructed some of the key aspects of the human pulmonary alveoli, including the microarchitecture, the ECM microenvironment, the ALI, and the mechanical breathing events. The alveolus in the lung is one of the most important structural determinants of the architecture of the respiratory parenchyma and lung functions. Our breathing alveolar lung-on-a-chip model could broadly reproduce the 3D anatomical structures and functions of the alveoli through the use of a precisely defined 3D porous GelMA hydrogel based on an inverse opal structure, while the breathing function was realized by situating the hAEC-populated GelMA structure in PDMS chip device. Based upon the breathing alveolar lung-on-chip model, we subsequently built a cigarette-smoking alveolar lung-on-a-chip platform, where the negative effects of smoking on the hAECs could be readily explored. We further demonstrated the applicability of our alveolar lung model for investigating SARS-COV-2 pseudoviral infection and treatment efficacies of antiviral drugs such as hydroxychloroquine, remdesivir, and amodiaquine. While remdesivir and amodiaquine have been approved by the Food and Drug Administration to treat COVID-19, in vitro and in vivo studies have shown the discrepancies in antiviral activity of hydroxychloroquine against SARS-COV-2.

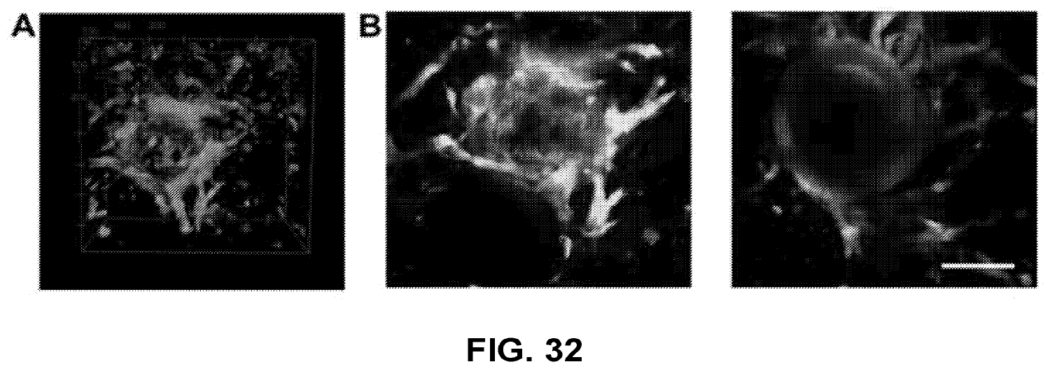

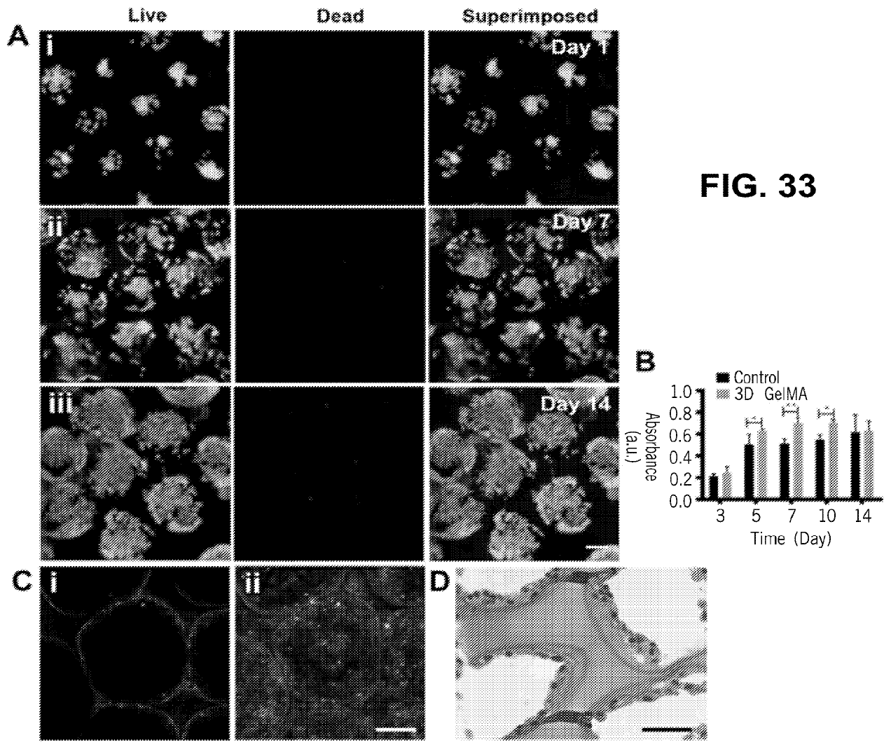

We have also showcased the possibility of creating the alveolar-capillary interface, which is another key microstructure in the human distal lung that enables the gas exchange in the alveoli by incorporating the human umbilical vein endothelial cells into the matrix of the GelMA inverse opal hydrogel to form the microvascular network surrounding the sacs (FIG. 32). In addition, to investigate the capability of our strategy to reconstitute pathological models of the lung or in particular, lung cancer, we also built a nonsmall-cell lung cancer (NSCLC) model with A549 cells. As shown in FIG. 33, the A549 cells could form a good alveolar structure in our platform with good cell viability and morphologies (note the multilayered growth of the A549 lung adenocarcinoma cells in some regions in comparison with the monolayer epithelium formed by normal hAECs).

Although surfactant lining layer at the ALI is extremely important for maintaining the alveolar functions, hAECs that we used in this study were commercially sourced, where the vendor unfortunately did not provide any information on AEC types I/II. It would be indeed very interesting for us in our future work to introduce defined populations of hAECs in our model, to study their effects on alveolar functions and pathologies such as infections.

While our system has some limitations, this example of the miniature alveolar lung-on-a-chip platform provides the foundation and can be readily expanded in the future to provide full functionality of the alveolar units in vitro to provide a niche for lung and lung disease modeling and research.

### Synthesis of Alginate Microbeads

Alginate and calcium chloride (Sigma-Aldrich, USA) were used to prepare 1-3% (w/v) alginate and 3% (w/v) crosslinking agent solutions, respectively. All experiments were carried out at a temperature of 20° C. and a humidity of 50%. In the experimental setup shown in FIG. 11, the alginate solution was pumped into a stainless-steel nozzle using a syringe pump (S-EF-SP, SKE Research Equipment, Italy) at a rate of 0.5 mL h⁻¹. The nozzle was mounted 20 cm above the calcium chloride bath and connected with the positive electrode. The ring electrode was grounded. Alginate was dripped into the calcium chloride bath where crosslinked alginate microbeads were formed. To investigate the factors on the sphericity and size of the microbeads produced, the parameters including alginate concentration, electric voltage, diameter of the nozzle, flow rate, and ring diameter were carefully studied.

### Synthesis of GelMA

GelMA was synthesized by the reaction of porcine skin gelatin (Type A, ˜300 g bloom, average Mw=90,000 Da, Sigma-Aldrich) with methacrylate anhydride (Sigma-Aldrich) at 50° C. for 2 h in phosphate-buffered saline (PBS, PH=7.4, ThermoFisher, USA). Methacrylic anhydride was added dropwise at a 0.2:1 weight ratio to gelatin under vigorously stirring. Following a 2× dilution with pre-heated PBS, the solution dialyzed against deionized water at 40° C. for 6 days with dialysis membrane (12,000-14,000 Da Mw cut-off, ThermoFisher). After dialysis, the GelMA solution was filtered with syringe filters (0.22 μm in pore size, VWR International, USA) and freeze-dried to yield a white solid for storage.

### Fabrication of GelMA Inverse Opal Structures

Alginate microbeads were closely packed in a PDMS (Dow Corning, USA) mold with the designated shape. Then, 7% (w/w) GelMA aqueous solution containing 0.5% (w/w) photoinitiator (2-hydroxy-1-[4-(2-hydroxyethoxy)phenyl]-2-methyl-1-propanone, Irgacure 2959, CIBA Chemicals, Switzerland) and 0.05% (w/w) mTG (100 U g⁻¹, Ajinomoto, Japan) was infiltrated by adding on top of the cubic close-packed alginate microbeads. The GelMA was subsequently photo-crosslinked under UV irradiation (˜10 mW cm⁻², 360-480 nm, OmniCure, USA) for 60 s and kept at 37° C. for 4 h to further allow mTG crosslinking. The resulting alginate microbeads/GelMA hydrogel composites were incubated in 0.01-M EDTA solution for up to 1 h at 37° C. under gentle shaking to remove alginate microbeads. The resulting GelMA was washed with excess PBS for five times with 5 min each and stored in PBS at 4° C. until use. The surface areas within the 3D GelMA inverse opal structures were calculated to be approximately 2 cm² each. The 2D scaffolds with a ˜0.8 cm² planar surface area and a dimension of 8 mm×10 mm×3 mm were used as controls.

Fabrication of the GelMA inverse opal structure-embedded chip device

The fabrication of alveolar lung-on-a-chip is schematically shown in FIG. 27. The upper and lower layers of the PDMS chip (FIG. 27(subpanel i)) were fabricated by casting the PDMS precursor (with the base to curing agent at a weight ratio of 11:1) on the molds prepared by laser-cut poly(methyl methacrylate) (PMMA) and then cured overnight at 80° C. The upper layer included two vacuum chambers separated by a cavity (8 mm wide×10 mm long×3 mm high). The lower layer contains five fluidic channels (1 mm wide×1 mm high) for medium flow, which was bonded (FIG. 27(subpanel ii)) to the upper layer by using plasma treatment. After the bonding, the chip was connected by tubing for vacuum application and medium perfusion (FIG. 27(subpanel iii)).

The PDMS chip was subsequently modified for bonding with the GelMA inverse opal hydrogel structure. Specifically, the PDMS chip was treated with oxygen plasma for 50 s followed by a solution of H₂O/H₂O₂/HCl (volume ratio at 5:1:1) for 5 min. After washing by deionized water, the chip was immersed with a solution of 1.5% (v/v) 3-(trimethoxysilyl) propyl methacrylate (TMSPMA) in ethanol/H₂O (volume ratio at 1:1). The PDMS chip was then cleaned with deionized water and dried with N₂ gas. After the modification, the channels at the bottom of the chip were filled with 1.5% (w/w) alginate solution and crosslinked with 3% (w/w) calcium chloride (FIG. 27(subpanel iv)). Afterwards, 150 μL of 7% GelMA solution was dropped into the PDMS chamber to bedspread the bottom as a thin layer and photocrosslinked by UV for 60 s (FIG. 27(subpanel v)). After the crosslinking of the GelMA base in the PDMS chip, the microbeads were packed on the top of this GelMA base (FIG. 27(subpanel vi)). Then after, ˜30 μL of the 7% GelMA precursor was dropped onto the chamber gently and allowed to diffuse through the spaces between microbeads in such a way that the topmost layer of the microbeads was not entirely submerged in GelMA. The entire structure was then exposed under UV to photocrosslink the GelMA, followed by chemical crosslinking with mTG (FIG. 27(subpanel vii)). The microbeads were subsequently removed by 0.01-M EDTA (FIG. 27(subpanel viii)) and, as such, the 3D inverse opal structure in the chip was fabricated.

### Fabrication of the Smoking Device

The smoke experiment was conducted following protocols laid out in previous studies, where these parameters were set up according to the smoking behavior that is observed in humans. In brief, to mimic the behaviors of a typical smoker, the smoking device was allowed to hold up to ten cigarettes (3R4F reference cigarettes). The particle size of the smoke produced by 3R4F reference cigarettes was reported to range in 14.6-685.4 nm with a count median diameter of 250 nm. By programing the controlling software, 12 puffs and a 2-s puff duration with a 22-s inter-puff interval were applied to each cigarette, while each puff was realized by drawing the air through the cigarette to a 50-ml syringe thus producing smoke in the smokebox. With this smoking device, the ten cigarettes could be finished in approximately 75 min. The smoke was introduced into the chips with a tubing containing a unidirectional check-valve connected to the smokebox. Autonomous “breathing” of the chips enabled effective smoking.

### Cell Culture in 3D GelMA Inverse Opal Structures

Primary hAECs (Cell Biologics, USA) were expanded in gelatin-coated T75 tissue culture flasks and cultured using the epithelial cell growth kit (H6621, Cell Biological) until 70-80% confluency. The hAECs (2×10⁵ cells in 1 mL of medium) were seeded into each GelMA inverse opal structure. After allowing the cells to settle for 10 min at the static condition, the scaffolds were placed on a shaker (40 rpm) for 3 h to facilitate uniform cell attachment. Loosely attached cells were gently washed off with fresh medium. The cells were cultured at the static condition and medium exchange was performed once every day. Towards the end of 14 days, the culture was supplemented with 1 μM of dexamethasone (Sigma-Aldrich) in the medium for 2 additional days to promote the tight junction formation.

### Cell Culture on 2D GelMA Surfaces

Similar to 3D GelMA inverse opal structures, after the 70-80% confluency in the T75 tissue culture flask, the hAECs (2×10⁵ cells in 1 mL of medium) were seeded on each 2D GelMA surfaces. The cells on the 2D GelMA surfaces were then cultured under static condition without any shaking. Afterwards, the cells on 2D surface were cultured under the similar conditions as for 3D structures.

### Construction of the Alveolar Lung-On-a-Chip

The cell seeding procedure remained the same as detailed above. Once seeded, the chips were cultured initially under a static condition with the medium both covering the entire hydrogel structures and flowing underneath through the channels at 60 μL h⁻¹ using a 205S series peristaltic pump (Watson-Marlow Pumps, USA). Towards the end of 14 days as the hAECs reached the confluence, the medium was replaced by that contained 1 μM of dexamethasone and cultured for another 2 days to promote the tight junction formation. After that, the medium immersing the hydrogel structure in each chip was carefully removed using a syringe needle to create the ALI. To mimic the physiological respiration events of the human lung, the two side breathing chambers were connected to the computer-controlled negative pressures to apply cyclic strains (10%) at 0.3 Hz, for 2 days.

### Cellular Characterizations

Cell viability was measured by the LIVE/DEAD® viability/cytotoxicity kit (ThermoFisher). The constructs were washed with PBS twice and placed individually in the wells of a 24-well plate. Next, a working solution containing 2 μL mL⁻¹ of ethidium homodimer-1 and 0.5 μL mL⁻¹ of calcein-AM in PBS was added. The constructs were incubated at 37° C. in the incubator for 30 min. Finally, the constructs were washed with PBS twice and resuspended in PBS for fluorescence imaging. The numbers of live and dead cells were counted using Image J (National Institutes of Health, USA).

Proliferation of hAECs was measured by the PrestoBlue™ reagent (ThermoFisher) according to the manufacturer's instructions. The constructs were washed once with PBS and placed individually in the wells of a 24-well plate, where a working solution which included the culture medium and the reagent at a proportion of 9:1 was added into the wells. After incubating for 2 h at 37° C., the supernatants were read by a spectrophotometer (excitation: 570 nm, emission: 600 nm; I-control, Tecan, Switzerland) to quantify.

For morphological analysis, Alexa 488-phalloidin and 4′,6-diamidino-2-phenylindole (DAPI) (both from ThermoFisher) for F-actin/nuclei staining were used. The constructs were washed with PBS twice, fixed with 4% (v/v) paraformaldehyde (PFA, Sigma-Aldrich) for 15 min, and washed gently for three times. Next, the constructs were permeabilized with 0.1% (v/v) Triton X-100 (Sigma-Aldrich) for 2 h at room temperature. Alexa 488-phalloidin diluted at a ratio of 1:40 (v/v) in 0.1% (w/w) bovine serum albumin (BSA, Sigma-Aldrich) in PBS and DAPI diluted at 1:1000 (v/v) in PBS served as the work solutions. Then constructs were washed once, and the prepared phalloidin staining solution was added and incubated for 45 min at 37° C. Finally, the constructs were washed with PBS and then stained with the DAPI working solution and incubated at 37° C. for 5 min.

For histological assessment, the constructs were washed with PBS three times and fixed with 4% (v/v) PFA for 48 h at 4° C., dehydrated, embedded in paraffin (ThermoFisher), and then serially sectioned (4 μm in thickness). The sections were mounted on glass slides, followed by de-paraffinization and staining with HE for observation of cell morphology.

For immunofluorescence staining, the ZO-1 antibody was used for the characterization of the tight junctions, while 4-HNE was utilized as a marker of oxidative stress to characterize the cells in chips after smoking. The constructs were washed with PBS three times, fixed with 4% (v/v) PFA for 15 min, and washed gently three times with PBS. Next, the constructs were permeabilized with 0.1% (v/v) Triton X-100 for 2 h at room temperature and blocked using PBS containing 1% (v/v) BSA and 5% (v/v) goat serum (ThermoFisher) for 30 min at room temperature, before incubating overnight at 4° ° C. with the ZO-1 antibody (1:200 (v/v), Abcam, USA) or 4-HNE antibody (5 μg mL⁻¹, R & D System, USA). These steps were followed by washing with PBS three times each for 5 min. Secondary antibody (ThermoFisher) in 1% (v/v) BSA was incubated for 2 h at room temperature to visualize ZO-1 or 4-HNE.

### Scanning Electron Microscopy Imaging

Scanning electron microscopy (JSM-7100F, JEOL, Japan) was used to characterize GelMA inverse opal structures as well as the morphologies of the hAECs grown inside. The samples were fixed with 4% (v/v) formaldehyde for 15 min, washed in PBS for three times, and dehydrated in a graded ethanol series for at least 10 min each. Subsequently, these samples were sputter-coated with platinum for 60 s. Images were taken at an accelerating voltage of 5 kV.

### Apoptosis Analyses

Annexin assay was conducted by using an annexin V-FITC apoptosis detection kit (ThermoFisher). The constructs were gently washed for three times and detached by immersing in 0.25% (v/v) trypsin (without EDTA, ThermoFisher) for 10-15 min. Afterwards, the cells were collected in the fresh medium containing 10% (v/v) FBS. The cells were then suspended in 1× binding buffer and stained with annexin V-FITC and PI for 10 min. Fluorescence-activated cell sorting (FACS) analysis was performed on a flow cytometer (BD FACSCalibur™, BD Biosciences, USA).

The caspase-3/7 analysis was realized by using caspase-3/7 green detection reagent (ThermoFisher). The constructs were gently washed for three times. After the addition of the caspase-3/7 working solution (8 μM in 5% (v/v) FBS) and incubation at 37° C. for 35 min, the constructs were fixed with 3.7% (v/v) formaldehyde for 15 min. Then, the cells were stained with a DAPI working solution for 5 min for the nuclei and imaged. The green fluorescence intensities and areas were quantified by ImageJ.

### Microarray Analyses

Total RNA was extracted from hAECs using a RNeasy micro kit (QIAGEN, Germany). The quality check and quantitation of RNA were performed by using Qubit fluorometer (ThermoFisher) and an Agilent 2100 Bioanalyzer (Agilent Technologies, USA). Paired-end libraries were prepared from total RNA using the TruSeq® RNA Sample Preparation Kit (Illumina, USA) according to the manufacturer's instructions. Purified libraries were quantified by the Qubit® 2.0 Fluorometer (ThermoFisher) and validated by the Agilent 2100 Bioanalyzer to confirm the insert sizes and calculate the molar concentrations. The clusters were generated by cBot with the library diluted to 10 pM and then sequenced on the Illumina HiSeq X-ten (Illumina, USA). Sequencing raw reads were pre-processed by filtering out rRNA reads, sequencing adapters, short-fragment reads, and other low-quality reads, as well as clean reads, and were mapped to the human genome (human GRCh38) using Hisat 2 (version: 2.0.4). We also screened differentially expressed genes between the groups with using Stringtie (version: 1.3.0) and edgeR. For GO and KEGG pathway enrichment analyses, all differentially expressed genes were mapped to terms in the KEGG and GO databases and queried for significantly enriched terms. K-means clustering, associated GO, and KEGG pathways were done with the R package cluster.

### Analysis of Chemokines and Cytokines

To compare chemokines and cytokines (IL-8, IL-6, IL-1b, MCP-1, and GM-CSF) secreted by hAECs in the chips under different conditions, the media (600 μL each) were collected and analyzed. The expression levels of these chemokines and cytokines were measured by Eve Technologies (Canada).

### Computational Simulations

To analyze the fluid dynamics within the designed system and evaluate the effect of structure stretching on air flow, computational simulations were performed using COMSOL Multiphysics (COMSOL Inc., Burlington, MA, USA). A representative region was extracted from the entire structure to reduce the global computational cost. Then, the symmetries present in the extracted region were used to further reduce the dimensions of the simulated model. The stretching of the structure was simulated as a sinusoidal function:

s(t)=|S sin(ωt)|

where s(t) is the value of stretching over time, S is the amplitude of the function equal to 10% of the structure width, and ω is the angular frequency equal to 0.63 rad s⁻¹.

The pressure generated by volume deformation was simulated as a sinusoidal function:

p(t)=P sin(ωt)

where p(t) is the value of pressure over time, P is the amplitude of the function equal to 1.33 Pa, and ω is the angular frequency equal to 1.26 rad s⁻¹.

Air was selected among the materials available in the software database and assigned to the fluid domain. A laminar flow physics was used to simulate the air flow inside the fluid domain. Initial values of velocity field and pressure were set to zero. Symmetry boundary conditions were set on symmetrical planes. A no-slip condition was set on system walls, constraining the fluid velocity to zero. An open boundary was set on top surface, while a pressure constrain was set on a point located in the middle of the simulated model, where the pressure followed the described equation.

To simulate the stretching of the structure over time the reported equation was applied to the lateral surface, while in the symmetry surface opposite to the stretched surface a null displacement was set. All the other surfaces were free to move accordingly. A free tetrahedral mesh was generated setting the mesh element size to normal.

A time-dependent study was performed using a time step equal to 0.625 s, and simulating a total time of 5 s. The distribution of flow streamlines entering the model from the top surface was analyzed over time. In addition, the direction of the flow over time was assessed evaluating the velocity vectors and velocity magnitude on a symmetry plane. To investigate the influence of the presented structure on air flow features a control group was simulated considering a system devoid of alveolar-like architecture.

### SARS-COV-2 Pseudotyped Virus Production and Infection of the Alveolar Lung-On-a-Chip

5×10⁵ HEK293T cells per well were seeded into 6-well plates. After 24 h of incubation at 37° C., 5% CO₂, the cells were transfected with 1.0 μg of pCMV3-SARS-COV2-Spike (the plasmid expressing the spike protein of SARS-COV-2, Sino Biological), 1.0 μg of pNL4-3 mCherry Luciferase (Addgene), and 0.5 μg of pAdvantage (Promega) with the TransIT-X2 transfection reagent (Abcam) to produce SARS-COV-2 pseudoviral particles (pCoV-VPs) according to the manufacturer's instructions. At 48 h post-transfection, the supernatants containing pCoV-VPs were collected and centrifuged at 10,000 g for 5 min to remove floating cells and cell debris. The culture supernatant containing pCoV-VPs was concentrated using a poly(ethylene glycol) virus precipitation kit (Abcam) according to the manufacturer's instructions and stored at 80° C. until use. The number of pCoV-VPs in the host cells was measured by luciferase activity at 48 h post-infection using the Bright-Glo reagent (Promega) according to the manufacturer's instructions.

To investigate pCoV-VP infection on alveolar lung-on-a-chip, the chips were exposed with amodiaquine (5 μM, Sigma-Aldrich), remdesivir (10 μM, MedKoo Biosciences), or hydroxychloroquine (40 μM, Sigma-Aldrich) 48 h prior to infection with pCoV-VPs, through the culture medium. The pCoV-VPs were exposed to the alveolar lung-on-a-chip also through the medium at multiplicity of infection of 0.5 and incubated for 48 h at 37° C. At 48 h post infection, the pCoV-VPs infection was observed under an Eclipse Ti2 inverted microscope (Nikon). pCoV-VP-mediated cytopathic effects were assessed by live/dead assay and MTS (Promega) assay according to the manufacturer's instruction. Uninfected alveolar lung-on-chips served as positive controls.

### Statistical Analyses

Statistical analyses were conducted by the GraphPad Prism 8 (USA) and SPSS statistics 26.0 (IBM, USA) software. All results and error bars are presented as means±standard deviations. Significance tests were performed using the student's t-test. Results were considered statistically significant for p<0.05, p<0.01, or p<0.001. All samples used for quantifications had n=3, and at least three randomly selected images were taken per sample for analyses.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A lung model apparatus, comprising:
an elastic hydrogel disposed within a housing,
the hydrogel comprising a plurality of sacs; and
a pressure chamber disposed within the housing and adjacent to the hydrogel,
the pressure chamber coupled to the hydrogel such that changes in pressure within the pressure chamber cause deformation of the hydrogel.

2. The apparatus of claim 1, wherein each sac of the plurality of sacs comprises an open space within the hydrogel.

3. The apparatus of claim 2, wherein a subset of the plurality of sacs of the hydrogel are interconnected by one or more openings in the hydrogel between adjacent sacs.

4. The apparatus of claim 1, further comprising a fluidic chamber disposed within the housing and adjacent to the hydrogel.

5. The apparatus of claim 4, wherein the fluidic chamber comprises a plurality of fluidic channels adjacent to the hydrogel.

6. The apparatus of claim 5, wherein the fluidic channels comprise culture media.

7. The apparatus of claim 1, further comprising a plurality of cells associated with the hydrogel.

8. The apparatus of claim 7, wherein the plurality of cells are adhered to surfaces of the plurality of sacs.

9. The apparatus of claim 8, wherein the plurality of cells comprises human alveolar epithelial cells (hAECs).

10. The apparatus of claim 7, wherein the plurality of cells is embedded within the hydrogel.

11. The apparatus of claim 10, wherein the plurality of cells embedded within the hydrogel comprise endothelial cells.

12. The apparatus of claim 1, wherein the hydrogel comprises a low-stiffness gelatin-based biomaterial.

13. The apparatus of claim 12, wherein the hydrogel comprises gelatin methacryloyl (GelMA).

14. The apparatus of claim 1, wherein the pressure chamber is configured to generate negative pressure, wherein the negative pressure causes the hydrogel to expand in at least one dimension.

15. The apparatus of claim 14, wherein the pressure chamber is configured to generate negative pressure in a cyclic manner.

16. The apparatus of claim 1, wherein the pressure chamber is a first pressure chamber, and wherein the apparatus further comprises a second pressure chamber disposed within the housing,
wherein the first and second pressure chambers are coupled to opposite sides of the hydrogel.

17. The apparatus of claim 16, wherein each of the first and second pressure chambers is configured to generate negative pressure, wherein the negative pressure in the first and second pressure chambers causes the hydrogel to expand in at least one dimension.

18. The apparatus of claim 17, wherein the first and second pressure chambers are configured to generate negative pressure in a cyclic manner.

19. The apparatus of claim 1, wherein the plurality of sacs comprises an inverse opal structure.

20. The apparatus of claim 19, wherein the inverse opal structure is formed from a plurality of beads assembled into a lattice.

21. The apparatus of claim 1, wherein the housing comprises a planar support structure having an opening therein,
wherein the pressure chamber is disposed within the housing adjacent to the opening, and
wherein the hydrogel is disposed within the opening in contact with the pressure chamber.

22. The apparatus of claim 21, wherein the pressure chamber is a first pressure chamber, and wherein the apparatus further comprises a second pressure chamber disposed within the housing adjacent to the opening,
wherein the second pressure chamber is disposed on an opposite side of the opening from the first pressure chamber, and
wherein the first and second pressure chambers are coupled to opposite sides of the hydrogel.

23. The apparatus of claim 22, wherein each of the first and second pressure chambers is configured to generate negative pressure, wherein the negative pressure in the first and second pressure chambers causes the hydrogel to expand in at least one dimension.

24. The apparatus of claim 23, wherein the first and second pressure chambers are configured to generate negative pressure in a cyclic manner.

25. The apparatus of claim 24, wherein the fluidic chamber comprises a plurality of fluidic channels adjacent to the hydrogel.

26. The apparatus of claim 25, wherein each of the first and second pressure chambers comprise pressure tubing coupled thereto, wherein the pressure tubing is coupled to a pressure source, and
wherein and the fluidic chamber comprises fluid tubing coupled thereto, wherein the fluid tubing is coupled to a fluid source.

27. The apparatus of claim 26, wherein the pressure source is configured to provide at least one of negative pressure or positive pressure to the pressure chamber, and
wherein the fluid source is configured to circulate culture media through the plurality of fluidic channels.

28. A method of making a lung model, comprising:
disposing a plurality of beads into an opening in a housing;
pouring a hydrogel-precursor into the opening of the housing within an interstitial space of the plurality of beads;
crosslinking the hydrogel-precursor to produce a hydrogel; and
dissolving the beads to produce an elastic hydrogel structure comprising a plurality of sacs.

29. The method of claim 28, wherein disposing a plurality of beads into an opening in a housing further comprises:
assembling the plurality of beads into a lattice pattern within the opening in the housing.

30. The method of claim 29, wherein the plurality of beads comprises alginate, and
wherein assembling the plurality of beads into the lattice pattern further comprises:
assembling the plurality of alginate beads into the lattice pattern within the opening in the housing.

31. The method of claim 30, wherein the hydrogel comprises a photoinitiator compound, and
wherein crosslinking the hydrogel further comprises:
crosslinking the hydrogel by applying at least one of visible or ultraviolet light to the hydrogel.

32. The method of claim 31, wherein the hydrogel further comprises a gelatin-based biomaterial and microbial transglutaminase (mTG), and
wherein crosslinking the hydrogel further comprises:
crosslinking the gelatin-based biomaterial using mTG.

33. The method of claim 32, wherein dissolving the beads further comprises:
dissolving the beads by applying a chelator to the beads.

34. The method of claim 28, wherein, prior to disposing a plurality of beads into an opening in a housing, the method comprises:
adding a dissolvable material to a base of the housing within the opening;
disposing a layer of hydrogel material over the dissolvable material; and
crosslinking the layer of hydrogel material,
wherein the plurality of beads is disposed onto the layer of crosslinked hydrogel material.

35. The method of claim 34, wherein the dissolvable material comprises alginate, and
wherein dissolving the beads to produce an elastic hydrogel structure comprising a plurality of sacs further comprises:
dissolving the layer of dissolvable material using a chelator to form a fluidic channel adjacent to the elastic hydrogel structure comprising the plurality of sacs.

36. The method of claim 35, wherein forming a fluidic channel adjacent to the elastic hydrogel structure comprising the plurality of sacs further comprises:
coupling the fluidic channel to a fluid source,
wherein the fluid source is configured to circulate culture media through the plurality of fluidic channels.

37. The method of claim 35, wherein forming a fluidic channel adjacent to the elastic hydrogel structure comprising the plurality of sacs further comprises:
forming a plurality of parallel fluidic channels adjacent to the elastic hydrogel structure comprising the plurality of sacs.

38. The method of claim 28, wherein, prior to disposing a plurality of beads into an opening in a housing, the method comprises:
providing the housing comprising at least one pressure chamber disposed within the housing adjacent to the opening,
wherein the at least one pressure chamber is coupled to the elastic hydrogel structure; and
coupling the at least one pressure chamber to a pressure source,
wherein the pressure source is configured to provide at least one of negative pressure or positive pressure to the at least one pressure chamber to cause deformation of the elastic hydrogel structure.

39. The method of claim 38, wherein coupling the at least one pressure chamber to a pressure source further comprises:
coupling the at least one pressure chamber to a pressure source configured to provide at least one of negative pressure or positive pressure to the at least one pressure chamber in a cyclic manner.

40. The method of claim 28, wherein, prior to disposing a plurality of beads into an opening in a housing, the method comprises:
providing the housing comprising a plurality of pressure chambers disposed within the housing adjacent to the opening,
wherein the plurality of pressure chambers are coupled to the elastic hydrogel structure, and
wherein at least two of the plurality of pressure chambers are disposed on opposite sides of the housing; and
coupling the plurality of pressure chambers to a pressure source,
wherein the pressure source is configured to provide at least one of negative pressure or positive pressure to the plurality of pressure chambers to cause deformation of the elastic hydrogel structure.

41. The method of claim 40, wherein coupling the plurality of pressure chambers to a pressure source further comprises:
coupling the plurality of pressure chambers to a pressure source configured to provide at least one of negative pressure or positive pressure to the at least one pressure chamber in a cyclic manner.

42. The method of claim 28, wherein dissolving the beads to produce an elastic hydrogel structure comprising a plurality of sacs further comprises:
dissolving the beads to produce the elastic hydrogel structure comprising a plurality of sacs,
wherein a subset of the plurality of sacs of the elastic hydrogel structure are interconnected by one or more openings in the elastic hydrogel structure between adjacent sacs.

43. The method of claim 28, further comprising:
associating a plurality of cells with the elastic hydrogel structure comprising the plurality of sacs.

44. The method of claim 43, wherein associating a plurality of cells with the elastic hydrogel structure comprising the plurality of sacs further comprises:
applying a plurality of human alveolar epithelial cells (hAECs) to a surface of the elastic hydrogel structure comprising the plurality of sacs.

45. The method of claim 44, further comprising exposing the hAECs to an airborne material comprising at least one of cigarette smoke or SARS-COV-2 virus, and analyzing an effect of the airborne material on the hAECs.

46. The method of claim 43, wherein associating a plurality of cells with the elastic hydrogel structure comprising the plurality of sacs further comprises, when pouring the hydrogel precursor into the opening of the housing:
embedding the plurality of cells within the hydrogel precursor, and
crosslinking the hydrogel precursor to produce the hydrogel comprising the plurality of cells embedded therein.

47. The method of claim 46, wherein embedding the plurality of cells within the hydrogel precursor further comprises:
embedding a plurality of structural and functional cells of the distal lung within the hydrogel precursor.

48. The method of claim 47, wherein embedding a plurality of structural and functional cells of the distal lung within the hydrogel precursor further comprises:
embedding at least one of endothelial cells, fibroblasts, or immune cells within the hydrogel precursor.
